SEQUENCE LISTING (343 sequences)

SEQ ID NO: 1 - protein, 15 aa, Homo sapiens
  MISC_FEATURE (1)..(1): amino acid with label
  MOD_RES (8)..(9): PHOSPHORYLATION
RPHFPQFSYSASGTA

SEQ ID NO: 2 - protein, 15 aa, Homo sapiens
  MISC_FEATURE (1)..(1): amino acid with label
  MOD_RES (8)..(8): PHOSPHORYLATION
RPHFPQFSYSASGTA

SEQ ID NO: 3 - protein, 9 aa, Homo sapiens
  MISC_FEATURE (9)..(9): amino acid with label
ERPQDVDQR

SEQ ID NO: 4 - protein, 17 aa, Homo sapiens
  MISC_FEATURE (17)..(17): amino acid with label
FYGAEIVSALDYLHSEK

SEQ ID NO: 5 - protein, 8 aa, Homo sapiens
  MISC_FEATURE (8)..(8): amino acid with label
SLLSGLLK

SEQ ID NO: 6 - protein, 10 aa, Homo sapiens
  MISC_FEATURE (10)..(10): amino acid with label
HTLDELNPQK

SEQ ID NO: 7 - protein, 7 aa, Homo sapiens
  MISC_FEATURE (7)..(7): amino acid with label
TYLLDFR

SEQ ID NO: 8 - protein, 10 aa, Homo sapiens
  MISC_FEATURE (10)..(10): amino acid with label
VPFLVAETPR

SEQ ID NO: 9 - protein, 8 aa, Homo sapiens
  MISC_FEATURE (8)..(8): amino acid with label
VVNPYYLR

SEQ ID NO: 10 - protein, 11 aa, Homo sapiens
  MISC_FEATURE (8)..(8): amino acid with label
GTELDCGIETD

SEQ ID NO: 11 - protein, 11 aa, Mus musculus
  MISC_FEATURE (11)..(11): amino acid with label
SGTDEEMACQK

SEQ ID NO: 12 - protein, 11 aa, Homo sapiens
  MISC_FEATURE (11)..(11): amino acid with label
SGTDVDAANLR

SEQ ID NO: 13 - protein, 11 aa, Homo sapiens
  MISC_FEATURE (11)..(11): amino acid with label
SGVDDDMACHK

SEQ ID NO: 14 - protein, 13 aa, Mus musculus
  MISC_FEATURE (13)..(13): amino acid with label
VGEVDVEQHTLAK

SEQ ID NO: 15 - protein, 23 aa, Mus musculus
  MISC_FEATURE (23)..(23): amino acid with label
VPVCEPEVELAEPEPEPELEHVR

SEQ ID NO: 16 - protein, 16 aa, Mus musculus
  MISC_FEATURE (16)..(16): amino acid with label
VPVTVTAASKPGLRPR

SEQ ID NO: 17 - protein, 13 aa, Homo sapiens
  MISC_FEATURE (13)..(13): amino acid with label
IGEVDVEQHTLAK

SEQ ID NO: 18 - protein, 17 aa, Homo sapiens
  MISC_FEATURE (17)..(17): amino acid with label
ISTLPQLNSALVQDLAK

SEQ ID NO: 19 - protein, 11 aa, Homo sapiens
  MISC_FEATURE (11)..(11): amino acid with label
NVVMVNQGLTK

SEQ ID NO: 20 - protein, 24 aa, Mus musculus
  MISC_FEATURE (22)..(22): amino acid with label
AAGGALQSTASLFVVSLSLLHLYS

SEQ ID NO: 21 - protein, 24 aa, Mus musculus
  MISC_FEATURE (22)..(22): amino acid with label
GGGSSLQSTAGLLALSLSLLHLYC

SEQ ID NO: 22 - protein, 16 aa, Homo sapiens
  MISC_FEATURE (16)..(16): amino acid with label
ESSETPDQFMTADETR

SEQ ID NO: 23 - protein, 13 aa, Homo sapiens
  MISC_FEATURE (13)..(13): amino acid with label
LVINSGNGAVEDR

SEQ ID NO: 24 - protein, 9 aa, Mus musculus
  MISC_FEATURE (9)..(9): amino acid with label
YAGVFHVEK

SEQ ID NO: 25 - protein, 12 aa, Homo sapiens
  MISC_FEATURE (12)..(12): amino acid with label
YGFIEGHVVIPR

SEQ ID NO: 26 - protein, 12 aa, Mus musculus
  MISC_FEATURE (12)..(12): amino acid with label
YGFIEGNVVIPR

SEQ ID NO: 27 - protein, 18 aa, Homo sapiens
  MISC_FEATURE (18)..(18): amino acid with label
DTANWLEINPDTGAISTR

SEQ ID NO: 28 - protein, 18 aa, Mus musculus
  MISC_FEATURE (18)..(18): amino acid with label
DTANWLEINPETGAIFTR

SEQ ID NO: 29 - protein, 14 aa, Mus musculus
  MISC_FEATURE (14)..(14): amino acid with label
DTGVISVLTSGLDR

SEQ ID NO: 30 - protein, 14 aa, Mus musculus
  MISC_FEATURE (14)..(14): amino acid with label
DTGVISVLTSGLDR

SEQ ID NO: 31 - protein, 10 aa, Homo sapiens
  MISC_FEATURE (10)..(10): amino acid with label
GLDARPEVTR

SEQ ID NO: 32 - protein, 7 aa, Homo sapiens
  MISC_FEATURE (7)..(7): amino acid with label
NMFTINR

SEQ ID NO: 33 - protein, 22 aa, Mus musculus
  MISC_FEATURE (12)..(12): amino acid with label
  MISC_FEATURE (22)..(22): amino acid with label
VFYSITGQGADKPPVGVFIIER

SEQ ID NO: 34 - protein, 22 aa, Homo sapiens
  MISC_FEATURE (22)..(22): amino acid with label
VFYSITGQGADTPPVGVFIIER

SEQ ID NO: 35 - protein, 13 aa, Mus musculus
  MISC_FEATURE (13)..(13): amino acid with label
VTDDDAPNTPAWK

SEQ ID NO: 36 - protein, 20 aa, Homo sapiens
  MISC_FEATURE (20)..(20): amino acid with label
YLPRPANPDEIGNFIDENLK

SEQ ID NO: 37 - protein, 14 aa, Homo sapiens
  MOD_RES (6)..(6): PHOSPHORYLATION
  MISC_FEATURE (14)..(14): amino acid with label
ILNDLSSDAPGVPR

SEQ ID NO: 38 - protein, 15 aa, Homo sapiens
  MOD_RES (7)..(7): PHOSPHORYLATION
  MISC_FEATURE (15)..(15): amino acid with label
KILNDLSSDAPGVPR

SEQ ID NO: 39 - protein, 28 aa, Homo sapiens
LFSGTQISTIAESEDSQESVDSVTDSQK

SEQ ID NO: 40 - protein, 10 aa, Homo sapiens
  MISC_FEATURE (10)..(10): amino acid with label
LHYGLPVVVK

SEQ ID NO: 41 - protein, 12 aa, Homo sapiens
  MISC_FEATURE (12)..(12): amino acid with label
LLNDEDQVVVNK

SEQ ID NO: 42 - protein, 14 aa, Homo sapiens
  MISC_FEATURE (14)..(14): amino acid with label
NEGVATYAAAVLFR

SEQ ID NO: 43 - protein, 9 aa, Homo sapiens
  MISC_FEATURE (9)..(9): amino acid with label
GLWIPEGEK

SEQ ID NO: 44 - protein, 11 aa, Mus musculus
  MOD_RES (9)..(9): PHOSPHORYLATION
  MISC_FEATURE (11)..(11): amino acid with label
GPTAENAEYLR

SEQ ID NO: 45 - protein, 11 aa, Homo sapiens
  MOD_RES (9)..(9): PHOSPHORYLATION
  MISC_FEATURE (11)..(11): amino acid with label
GSTAENAEYLR

SEQ ID NO: 46 - protein, 10 aa, Homo sapiens
  MISC_FEATURE (10)..(10): amino acid with label
IPLENLQIIR

SEQ ID NO: 47 - protein, 12 aa, Homo sapiens
  MOD_RES (10)..(10): S-carboxyamidomethylcysteine
  MISC_FEATURE (12)..(12): amino acid with label
NYVVTDHGSCVR

SEQ ID NO: 48 - protein, 11 aa, Homo sapiens
  MOD_RES (8)..(8): S-carboxyamidomethylcysteine
  MISC_FEATURE (11)..(11): amino acid with label
IAIWTTECENR

SEQ ID NO: 49 - protein, 14 aa, Homo sapiens
  MISC_FEATURE (14)..(14): amino acid with label
IVIGYQSHADTATK

SEQ ID NO: 50 - protein, 6 aa, Homo sapiens
  MOD_RES (3)..(3): PHOSPHORYLATION
  MISC_FEATURE (6)..(6): amino acid with label
SGSTTK

SEQ ID NO: 51 - protein, 8 aa, Homo sapiens
  MOD_RES (3)..(3): PHOSPHORYLATION
  MISC_FEATURE (8)..(8): amino acid with label
SGSTTKNR

SEQ ID NO: 52 - protein, 15 aa, Homo sapiens
  MISC_FEATURE (15)..(15): amino acid with label
TESNQEVANPEHYIK

SEQ ID NO: 53 - protein, 7 aa, Homo sapiens
  MISC_FEATURE (7)..(7): amino acid with label
WLITLNK

SEQ ID NO: 54 - protein, 26 aa, Homo sapiens
  MOD_RES (11)..(11): PHOSPHORYLATION
  MISC_FEATURE (26)..(26): amino acid with label
DLPTIPGVTSSSSDEPPMEASQSHLR

SEQ ID NO: 55 - protein, 15 aa, Mus musculus
DGGEFKLVDAEEVAR

SEQ ID NO: 56 - protein, 18 aa, Homo sapiens
  MOD_RES (7)..(7): PHOSPHORYLATION
  MISC_FEATURE (18)..(18): amino acid with label
DLELPLSPSLLGGPGPER

SEQ ID NO: 57 - protein, 13 aa, Mus musculus
EQGNGHIISWTSR

SEQ ID NO: 58 - protein, 9 aa, Homo sapiens
LVDAEEVAR

SEQ ID NO: 59 - protein, 12 aa, Homo sapiens
SEELNVEPGLGR

SEQ ID NO: 60 - protein, 11 aa, Homo sapiens
  MISC_FEATURE (11)..(11): amino acid with label
GIWIPDGENVK

SEQ ID NO: 61 - protein, 15 aa, Homo sapiens
  MISC_FEATURE (15)..(15): amino acid with label
LLDIDETEYHADGGK

SEQ ID NO: 62 - protein, 13 aa, Homo sapiens
  MISC_FEATURE (13)..(13)
LPASPETHLDMLR

SEQ ID NO: 63 - protein, 19 aa, Homo sapiens
  MOD_RES (9)..(9): PHOSPHORYLATION
  MOD_RES (13)..(13): PHOSPHORYLATION
  MOD_RES (15)..(15): PHOSPHORYLATION
  MISC_FEATURE (19)..(19): amino acid with label
IADPEHDHTGFLTEYVATR

SEQ ID NO: 64 - protein, 19 aa, Homo sapiens
  MOD_RES (13)..(13): PHOSPHORYLATION
  MOD_RES (15)..(15): PHOSPHORYLATION
  MISC_FEATURE (19)..(19): amino acid with label
IADPEHDHTGFLTEYVATR

SEQ ID NO: 65 - protein, 9 aa, Homo sapiens
  MISC_FEATURE (9)..(9): amino acid with label
NYLQSLPSK

SEQ ID NO: 66 - protein, 18 aa, Homo sapiens
  MOD_RES (10)..(10): S-carboxyamidomethylcysteine
  MISC_FEATURE (18)..(18): amino acid with label
SQQLSNDHICYFLYQILR

SEQ ID NO: 67 - protein, 9 aa, Homo sapiens
  MISC_FEATURE (9)..(9): amino acid with label
GQVFDVGPR

SEQ ID NO: 68 - protein, 9 aa, Homo sapiens
  MISC_FEATURE (9)..(9): amino acid with label
NYLLSLPHK

SEQ ID NO: 69 - protein, 19 aa, Homo sapiens
  MOD_RES (13)..(13): PHOSPHORYLATION
  MOD_RES (15)..(15): PHOSPHORYLATION
  MISC_FEATURE (19)..(19): amino acid with label
VADPDHDHTGFLTEYVATR

SEQ ID NO: 70 - protein, 10 aa, Homo sapiens
  MISC_FEATURE (10)..(10): amino acid with label
FFEILSPVYR

SEQ ID NO: 71 - protein, 10 aa, Homo sapiens
  MISC_FEATURE (10)..(10): amino acid with label
NLLDVIDQAR

SEQ ID NO: 72 - protein, 11 aa, Homo sapiens
  MOD_RES (9)..(9): S-carboxyamidomethylcysteine
  MISC_FEATURE (11)..(11): amino acid with label
NVLVSSNDCVK

SEQ ID NO: 73 - protein, 9 aa, Homo sapiens
  MOD_RES (7)..(7): PHOSPHORYLATION
  MOD_RES (8)..(8): PHOSPHORYLATION
  MISC_FEATURE (9)..(9): amino acid with label
YMEDSTYYK

SEQ ID NO: 74 - protein, 9 aa, Homo sapiens
  MOD_RES (7)..(7): PHOSPHORYLATION
  MISC_FEATURE (9)..(9): amino acid with label
YMEDSTYYK

SEQ ID NO: 75 - protein, 9 aa, Homo sapiens
  MOD_RES (3)..(3): PHOSPHORYLATION
  MISC_FEATURE (9)..(9): amino acid with label
AASMDNNSK

SEQ ID NO: 76 - protein, 12 aa, Homo sapiens
LTLSQIYEWMVK

SEQ ID NO: 77 - protein, 11 aa, Homo sapiens
  MOD_RES (6)..(6): PHOSPHORYLATION
  MOD_RES (9)..(9): PHOSPHORYLATION
  MISC_FEATURE (11)..(11): amino acid with label
TSSNASTISGR

SEQ ID NO: 78 - protein, 11 aa, Homo sapiens
  MOD_RES (9)..(9): PHOSPHORYLATION
  MISC_FEATURE (11)..(11): amino acid with label
TSSNASTISGR

SEQ ID NO: 79 - protein, 22 aa, Homo sapiens
WPASPGSHSNDDFDNWSTFRPR

SEQ ID NO: 80 - protein, 14 aa, Homo sapiens
  MISC_FEATURE (14)..(14): amino acid with label
ESESAPGDFSLSVK

SEQ ID NO: 81 - protein, 9 aa, Homo sapiens
  MISC_FEATURE (9)..(9): amino acid with label
FGNDVQHFK

SEQ ID NO: 82 - protein, 12 aa, Homo sapiens
  MISC_FEATURE (12)..(12): amino acid with label
FNSLNELVDYHR

SEQ ID NO: 83 - protein, 7 aa, Homo sapiens
  MISC_FEATURE (7)..(7): amino acid with label
YFLWVVK

SEQ ID NO: 84 - protein, 21 aa, Homo sapiens
  MOD_RES (1)..(1): PHOSPHORYLATION
  MOD_RES (3)..(3): PHOSPHORYLATION
  MOD_RES (8)..(8): S-carboxyamidomethylcysteine
  MISC_FEATURE (21)..(21): amino acid with label
TTSFAESCKPVQQPSAFGSMK

SEQ ID NO: 85 - protein, 17 aa, Homo sapiens
  MISC_FEATURE (17)..(17): amino acid with label
DIKPQNLLLDPDTAVLK

SEQ ID NO: 86 - protein, 9 aa, Homo sapiens
  MISC_FEATURE (9)..(9): amino acid with label
LLEYTPTAR

SEQ ID NO: 87 - protein, 11 aa, Homo sapiens
  MOD_RES (9)..(9): S-carboxyamidomethylcysteine
  MISC_FEATURE (11)..(11): amino acid with label
TPPEAIALCSR

SEQ ID NO: 88 - protein, 21 aa, Homo sapiens
  MOD_RES (3)..(3): PHOSPHORYLATION
  MOD_RES (8)..(8): S-carboxyamidomethylcysteine
  MISC_FEATURE (21)..(21)
TTSFAESCKPVQQPSAFGSMK

SEQ ID NO: 89 - protein, 9 aa, Homo sapiens
  MOD_RES (1)..(1): PHOSPHORYLATION
  MISC_FEATURE (9)..(9): amino acid with label
SAPATGGVK

SEQ ID NO: 90 - protein, 10 aa, Homo sapiens
  MOD_RES (2)..(2): PHOSPHORYLATION
  MISC_FEATURE (10)..(10): amino acid with label
KSAPATGGVK

SEQ ID NO: 91 - protein, 9 aa, Homo sapiens
  MISC_FEATURE (9)..(9): amino acid with label
WMALESILR

SEQ ID NO: 92 - protein, 15 aa, Homo sapiens
  MISC_FEATURE (15)..(15): amino acid with label
ITELMGYEPEELLGR

SEQ ID NO: 93 - protein, 10 aa, Homo sapiens
  MISC_FEATURE (10)..(10)
NLLQGEELLR

SEQ ID NO: 94 - protein, 12 aa, Homo sapiens
  MISC_FEATURE (12)..(12): amino acid with label
SPNVLSVALSQR

SEQ ID NO: 95 - protein, 14 aa, Homo sapiens
  MISC_FEATURE (14)..(14): amino acid with label
SSADPALNQEVALK

SEQ ID NO: 96 - protein, 22 aa, Mus musculus
  MOD_RES (8)..(8): S-carboxyamidomethylcysteine
  MOD_RES (17)..(17): PHOSPHORYLATION
  MISC_FEATURE (22)..(22): amino acid with label
ELELLFGCHVEGDAAETPPRPR

SEQ ID NO: 97 - protein, 22 aa, Homo sapiens
  MOD_RES (8)..(8): S-carboxyamidomethylcysteine
  MOD_RES (17)..(17): PHOSPHORYLATION
  MISC_FEATURE (22)..(22): amino acid with label
ELELMFGCQVEGDAAETPPRPR

SEQ ID NO: 98 - protein, 22 aa, Homo sapiens
  MOD_RES (2)..(2): S-carboxyamidomethylcysteine
  MOD_RES (13)..(13): PHOSPHORYLATION
  MOD_RES (17)..(17): PHOSPHORYLATION
  MISC_FEATURE (22)..(22): amino acid with label
LCDFGVSGQLIDSMANSFVGTR

SEQ ID NO: 99 - protein, 14 aa, Homo sapiens
  MOD_RES (1)..(1): PHOSPHORYLATION
  MOD_RES (6)..(6): S-carboxyamidomethylcysteine
  MISC_FEATURE (14)..(14): amino acid with label
TMDAGCKPYMAPER

SEQ ID NO: 100 - protein, 11 aa, Homo sapiens
  MISC_FEATURE (11)..(11): amino acid with label
DVKPSNVLINK

SEQ ID NO: 101 - protein, 14 aa, Homo sapiens
  MISC_FEATURE (14)..(14): amino acid with label
FPYESWGTPFQQLK

SEQ ID NO: 102 - protein, 11 aa, Homo sapiens
  MISC_FEATURE (11)..(11): amino acid with label
HAQSGTIMAVK

SEQ ID NO: 103 - protein, 17 aa, Homo sapiens
  MISC_FEATURE (17)..(17): amino acid with label
NFEVEADDLVTISELGR

SEQ ID NO: 104 - protein, 12 aa, Homo sapiens
  MISC_FEATURE (12)..(12): amino acid with label
KIEDASNPLLLK

SEQ ID NO: 105 - protein, 17 aa, Homo sapiens
  MISC_FEATURE (17)..(17): amino acid with label
SIGEAIQYLHSINIAHR

SEQ ID NO: 106 - protein, 10 aa, Homo sapiens
  MOD_RES (4)..(4): PHOSPHORYLATION
  MOD_RES (8)..(8): PHOSPHORYLATION
  MISC_FEATURE (10)..(10): amino acid with label
VPQTPLHTSR

SEQ ID NO: 107 - protein, 10 aa, Homo sapiens
  MOD_RES (4)..(4): PHOSPHORYLATION
  MISC_FEATURE (10)..(10): amino acid with label
VPQTPLHTSR

SEQ ID NO: 108 - protein, 13 aa, Homo sapiens
  MISC_FEATURE (13)..(13): amino acid with label
VTSQVLGLGINGK

SEQ ID NO: 109 - protein, 8 aa, Homo sapiens
  MISC_FEATURE (8)..(8): amino acid with label
IPEQILGK

SEQ ID NO: 110 - protein, 14 aa, Homo sapiens
  MISC_FEATURE (14)..(14): amino acid with label
ISELGAGNGGVVFK

SEQ ID NO: 111 - protein, 31 aa, Homo sapiens
  MISC_FEATURE (31)..(31): amino acid with label
KPTPIQLNPAPDGSAVNGTSSAETNLEALQK

SEQ ID NO: 112 - protein, 22 aa, Homo sapiens
  MOD_RES (2)..(2): S-carboxyamidomethylcysteine
  MOD_RES (17)..(17): PHOSPHORYLATION
  MISC_FEATURE (22)..(22): amino acid with label
LCDFGVSGQLIDSMANSFVGTR

SEQ ID NO: 113 - protein, 16 aa, Homo sapiens
  MISC_FEATURE (16)..(16): amino acid with label
LPSGVFSLEFQDFVNK

SEQ ID NO: 114 - protein, 12 aa, Homo sapiens
  MISC_FEATURE (12)..(12): amino acid with label
DVKPSNILVNSR

SEQ ID NO: 115 - protein, 14 aa, Homo sapiens
  MISC_FEATURE (14)..(14): amino acid with label
ISELGAGNGGVVTK

SEQ ID NO: 116 - protein, 11 aa, Homo sapiens
  MISC_FEATURE (11)..(11): amino acid with label
DLIGFGLQVAK

SEQ ID NO: 117 - protein, 13 aa, Homo sapiens
  MOD_RES (7)..(7): PHOSPHORYLATION
  MOD_RES (8)..(8): PHOSPHORYLATION
  MISC_FEATURE (13)..(13): amino acid with label
DMYDKEYYSVHNK

SEQ ID NO: 118 - protein, 8 aa, Homo sapiens
  MOD_RES (2)..(2): PHOSPHORYLATION
  MOD_RES (3)..(3): PHOSPHORYLATION
  MISC_FEATURE (8)..(8): amino acid with label
EYYSVHNK

SEQ ID NO: 119 - protein, 14 aa, Homo sapiens
  MISC_FEATURE (14)..(14): amino acid with label
GDLTIANLGTSEGR

SEQ ID NO: 120 - protein, 10 aa, Homo sapiens
  MISC_FEATURE (10)..(10): amino acid with label
LNSELNIEWK

SEQ ID NO: 121 - protein, 24 aa, Homo sapiens
  MOD_RES (1)..(1): PHOSPHORYLATION
  MISC_FEATURE (24)..(24): amino acid with label
TDSYSAGQSVEILDGVELGEPAHK

SEQ ID NO: 122 - protein, 11 aa, Homo sapiens
  MISC_FEATURE (11)..(11): amino acid with label
DASAVSLSESK

SEQ ID NO: 123 - protein, 16 aa, Homo sapiens
  MISC_FEATURE (16)..(16): amino acid with label
GPTPAILESLISINNK

SEQ ID NO: 124 - protein, 13 aa, Homo sapiens
  MISC_FEATURE (13)..(13): amino acid with label
IHGALLILNELVR

SEQ ID NO: 125 - protein, 26 aa, Homo sapiens
  MOD_RES (5)..(5): PHOSPHORYLATION
  MISC_FEATURE (26)..(26)
TRTDSYSAGQSVEILDGVELGEPAHK

SEQ ID NO: 126 - protein, 12 aa, Homo sapiens
  MISC_FEATURE (12)..(12): amino acid with label
VLGLLGALDPYK

SEQ ID NO: 127 - protein, 14 aa, Homo sapiens
  MISC_FEATURE (14)..(14): amino acid with label
ATAYILSVQAEEQK

SEQ ID NO: 128 - protein, 11 aa, Homo sapiens
  MISC_FEATURE (11)..(11): amino acid with label
DQIPELENNEK

SEQ ID NO: 129 - protein, 13 aa, Homo sapiens
  MISC_FEATURE (13)..(13): amino acid with label
FELLPTPPLSPSR

SEQ ID NO: 130 - protein, 13 aa, Mus musculus
  MISC_FEATURE (4)..(4): amino acid with label
  MISC_FEATURE (13)..(13): amino acid with label
GHSKPPHSPLVLK

SEQ ID NO: 131 - protein, 8 aa, Homo sapiens
  MISC_FEATURE (8)..(8): amino acid with label
LASYQAAR

SEQ ID NO: 132 - protein, 8 aa, Homo sapiens
  MISC_FEATURE (8)..(8): amino acid with label
DLLIDEWK

SEQ ID NO: 133 - protein, 15 aa, Homo sapiens
  MISC_FEATURE (15)..(15): amino acid with label
HENVIGLLDVFTPAR

SEQ ID NO: 134 - protein, 13 aa, Homo sapiens
  MOD_RES (7)..(7): PHOSPHORYLATION
  MOD_RES (9)..(9): PHOSPHORYLATION
  MISC_FEATURE (13)..(13): amino acid with label
HTDDEMTGYVATR

SEQ ID NO: 135 - protein, 15 aa, Homo sapiens
  MISC_FEATURE (15)..(15): amino acid with label
LTDDHVQFLIYQILR

SEQ ID NO: 136 - protein, 13 aa, Homo sapiens
  MISC_FEATURE (13)..(13): amino acid with label
TLFPGTDHIDQLK

SEQ ID NO: 137 - protein, 15 aa, Homo sapiens
  MOD_RES (1)..(1): S-carboxyamidomethylcysteine
  MOD_RES (2)..(2): PHOSPHORYLATION
  MISC_FEATURE (15)..(15): amino acid with label
CSDSDGLAPPQHLIR

SEQ ID NO: 138 - protein, 13 aa, Mus musculus
  MOD_RES (5)..(5): S-carboxyamidomethylcysteine
  MOD_RES (9)..(9): PHOSPHORYLATION
  MISC_FEATURE (13)..(13): amino acid with label
ALPTCTSASPPQK

SEQ ID NO: 139 - protein, 9 aa, Homo sapiens
  MISC_FEATURE (9)..(9): amino acid with label
ELNEALELK

SEQ ID NO: 140 - protein, 19 aa, Homo sapiens
  MISC_FEATURE (19)..(19): amino acid with label
RPILTIITLEDSSGNLLGR

SEQ ID NO: 141 - protein, 9 aa, Homo sapiens
  MISC_FEATURE (9)..(9): amino acid with label
TYQGSYGFR

SEQ ID NO: 142 - protein, 10 aa, Homo sapiens
  MISC_FEATURE (10)..(10): amino acid with label
HINWEELLAR

SEQ ID NO: 143 - protein, 10 aa, Homo sapiens
  MOD_RES (5)..(5): S-carboxyamidomethylcysteine
  MISC_FEATURE (10)..(10): amino acid with label
IRPECFELLR

SEQ ID NO: 144 - protein, 18 aa, Homo sapiens
  MISC_FEATURE (18)..(18): amino acid with label
LGAGPGDAGEVQAHPFFR

SEQ ID NO: 145 - protein, 19 aa, Homo sapiens
  MOD_RES (1)..(1): PHOSPHORYLATION
  MOD_RES (5)..(5): PHOSPHORYLATION
  MOD_RES (11)..(11): PHOSPHORYLATION
  MISC_FEATURE (19)..(19): amino acid with label
TPKDSPGIPPSAGAHQLFR

SEQ ID NO: 146 - protein, 19 aa, Homo sapiens
  MOD_RES (1)..(1): PHOSPHORYLATION
  MOD_RES (5)..(5): PHOSPHORYLATION
  MISC_FEATURE (19)..(19): amino acid with label
TPKDSPGIPPSAGAHQLFR

SEQ ID NO: 147 - protein, 22 aa, Homo sapiens
  MOD_RES (8)..(8): PHOSPHORYLATION
  MOD_RES (10)..(10): S-carboxyamidomethylcysteine
  MISC_FEATURE (22)..(22): amino acid with label
AENGLLMTPCYTANFVAPEVLK

SEQ ID NO: 148 - protein, 16 aa, Homo sapiens
  MOD_RES (2)..(2): PHOSPHORYLATION
  MOD_RES (8)..(8): PHOSPHORYLATION
  MISC_FEATURE (16)..(16): amino acid with label
DSPGIPPSAGAHQLFR

SEQ ID NO: 149 - protein, 16 aa, Homo sapiens
  MOD_RES (2)..(2): PHOSPHORYLATION
  MISC_FEATURE (16)..(16): amino acid with label
DSPGIPPSAGAHQLFR

SEQ ID NO: 150 - protein, 13 aa, Homo sapiens
  MISC_FEATURE (13)..(13): amino acid with label
DILADVNHPFVVK

SEQ ID NO: 151 - protein, 20 aa, Homo sapiens
  MOD_RES (18)..(18): S-carboxyamidomethylcysteine
  MISC_FEATURE (20)..(20): amino acid with label
DLKPSNILYVDESGNPECLR

SEQ ID NO: 152 - protein, 16 aa, Homo sapiens
  MISC_FEATURE (16)..(16): amino acid with label
FTLSGGNWNTVSETAK

SEQ ID NO: 153 - protein, 17 aa, Homo sapiens
  MOD_RES (3)..(3): PHOSPHORYLATION
  MISC_FEATURE (17)..(17): amino acid with label
GFSFVATGLMEDDGKPR

SEQ ID NO: 154 - protein, 11 aa, Homo sapiens
  MISC_FEATURE (11)..(11): amino acid with label
NLVFSDGYVVK

SEQ ID NO: 155 - protein, 12 aa, Homo sapiens
  MISC_FEATURE (12)..(12): amino acid with label
GAMAATYSALNR

SEQ ID NO: 156 - protein, 30 aa, Homo sapiens
  MOD_RES (3)..(3): PHOSPHORYLATION
  MISC_FEATURE (30)..(30): amino acid with label
GFSFVAITSDDESQAMQTVGVHSIVQQLHR

SEQ ID NO: 157 - protein, 8 aa, Homo sapiens
  MISC_FEATURE (8)..(8): amino acid with label
LTAALVLR

SEQ ID NO: 158 - protein, 12 aa, Homo sapiens
  MISC_FEATURE (12)..(12): amino acid with label
GDEVDGVDEVAK

SEQ ID NO: 159 - protein, 7 aa, Homo sapiens
  MISC_FEATURE (7)..(7): amino acid with label
GVDEVAK

SEQ ID NO: 160 - protein, 11 aa, Homo sapiens
  MISC_FEATURE (11)..(11): amino acid with label
TLGDFAAEYAK

SEQ ID NO: 161 - protein, 13 aa, Homo sapiens
  MISC_FEATURE (13)..(13): amino acid with label
TTNFAGILSQGLR

SEQ ID NO: 162 - protein, 15 aa, Homo sapiens
  MISC_FEATURE (15)..(15): amino acid with label
VVSEDFLQDVSASTK

SEQ ID NO: 163 - protein, 10 aa, Homo sapiens
  MISC_FEATURE (10)..(10): amino acid with label
AGNEYLIFQK

SEQ ID NO: 164 - protein, 20 aa, Mus musculus
  MISC_FEATURE (20)..(20): amino acid with label
AHPFFETITWENLHQQTPPK

SEQ ID NO: 165 - protein, 19 aa, Homo sapiens
  MOD_RES (3)..(3): PHOSPHORYLATION
  MISC_FEATURE (19)..(19): amino acid with label
ANSFVGTAQYVSPELLTEK

SEQ ID NO: 166 - protein, 16 aa, Homo sapiens
  MISC_FEATURE (16)..(16): amino acid with label
FYTAEIVSALEYLHGK

SEQ ID NO: 167 - protein, 18 aa, Homo sapiens
  MISC_FEATURE (18)..(18): amino acid with label
QLLLTEGPHLYYVDPVNK

SEQ ID NO: 168 - protein, 12 aa, Homo sapiens
  MISC_FEATURE (12)..(12): amino acid with label
LIESSLGSIATK

SEQ ID NO: 169 - protein, 13 aa, Homo sapiens
  MISC_FEATURE (13)..(13): amino acid with label
TAEDDETPVDLNK

SEQ ID NO: 170 - protein, 12 aa, Homo sapiens
  MISC_FEATURE (12)..(12): amino acid with label
TSSTLSEDQMSR

SEQ ID NO: 171 - protein, 9 aa, Homo sapiens
  MISC_FEATURE (9)..(9): amino acid with label
LINLTDILK

SEQ ID NO: 172 - protein, 10 aa, Homo sapiens
  MOD_RES (2)..(2): S-carboxyamidomethylcysteine
  MOD_RES (4)..(4): S-carboxyamidomethylcysteine
VCGCDEYFLE

SEQ ID NO: 173 - protein, 8 aa, Homo sapiens
  MISC_FEATURE (8)..(8): amino acid with label
FLLQHLGR

SEQ ID NO: 174 - protein, 10 aa, Homo sapiens
  MISC_FEATURE (10)..(10): amino acid with label
HESLAQYNAK

SEQ ID NO: 175 - protein, 18 aa, Homo sapiens
  MISC_FEATURE (18)..(18): amino acid with label
TATGFGFAEPYNLYGSLK

SEQ ID NO: 176 - protein, 9 aa, Mus musculus
  MOD_RES (5)..(5): PHOSPHORYLATION
  MISC_FEATURE (9)..(9): amino acid with label
AREGSFEAR

SEQ ID NO: 177 - protein, 9 aa, Homo sapiens
  MOD_RES (5)..(5): PHOSPHORYLATION
  MISC_FEATURE (9)..(9): amino acid with label
AREGSFESR

SEQ ID NO: 178 - protein, 18 aa, Homo sapiens
  MOD_RES (7)..(7): PHOSPHORYLATION
  MISC_FEATURE (18)..(18): amino acid with label
DLELPLSPSLLGGQGPER

SEQ ID NO: 179 - protein, 7 aa, Mus musculus
  MOD_RES (3)..(3): PHOSPHORYLATION
  MISC_FEATURE (7)..(7): amino acid with label
EGSFEAR

SEQ ID NO: 180 - protein, 7 aa, Homo sapiens
  MOD_RES (3)..(3): PHOSPHORYLATION
  MISC_FEATURE (7)..(7): amino acid with label
EGSFESR

SEQ ID NO: 181 - protein, 17 aa, Homo sapiens
  MOD_RES (8)..(8): S-carboxyamidomethylcysteine
  MISC_FEATURE (17)..(17): amino acid with label
GVLDVPACQIAIRPEGK

SEQ ID NO: 182 - protein, 15 aa, Homo sapiens
  MOD_RES (8)..(8): PHOSPHORYLATION
  MOD_RES (12)..(12): PHOSPHORYLATION
  MISC_FEATURE (15)..(15): amino acid with label
IGTAEPDYGALYEGR

SEQ ID NO: 183 - protein, 15 aa, Homo sapiens
  MOD_RES (8)..(8): PHOSPHORYLATION
  MISC_FEATURE (15)..(15): amino acid with label
IGTAEPDYGALYEGR

SEQ ID NO: 184 - protein, 15 aa, Homo sapiens
  MISC_FEATURE (15)..(15): amino acid with label
NNYSEDLELASLLIK

SEQ ID NO: 185 - protein, 15 aa, Homo sapiens
  MOD_RES (5)..(5): PHOSPHORYLATION
  MISC_FEATURE (15)..(15): amino acid with label
NPGFYVEANPMPTFK

SEQ ID NO: 186 - protein, 14 aa, Homo sapiens
  MISC_FEATURE (14)..(14): amino acid with label
SGDITYGQFAQLYR

SEQ ID NO: 187 - protein, 10 aa, Homo sapiens
  MOD_RES (6)..(6): PHOSPHORYLATION
  MISC_FEATURE (10)..(10): amino acid with label
DINSLYDVSR

SEQ ID NO: 188 - protein, 12 aa, Homo sapiens
  MISC_FEATURE (12)..(12): amino acid with label
GILDLNTYNVVK

SEQ ID NO: 189 - protein, 9 aa, Homo sapiens
  MOD_RES (3)..(3): S-carboxyamidomethylcysteine
  MISC_FEATURE (9)..(9): amino acid with label
HYCAIADAK

SEQ ID NO: 190 - protein, 15 aa, Homo sapiens
  MOD_RES (2)..(2): PHOSPHORYLATION
  MOD_RES (6)..(6): PHOSPHORYLATION
  MISC_FEATURE (15)..(15): amino acid with label
MYVDPSEINPSMPQR

SEQ ID NO: 191 - protein, 18 aa, Homo sapiens
  MOD_RES (11)..(11): PHOSPHORYLATION
  MISC_FEATURE (18)..(18): amino acid with label
QEELNNQLFLYDTHQNLR

SEQ ID NO: 192 - protein, 19 aa, Homo sapiens
  MOD_RES (12)..(12): PHOSPHORYLATION
  MISC_FEATURE (19)..(19): amino acid with label
RQEELNNQLFLYDTHQNLR

SEQ ID NO: 193 - protein, 10 aa, Homo sapiens
  MISC_FEATURE (10)..(10): amino acid with label
VDSSNYDPFR

SEQ ID NO: 194 - protein, 20 aa, Mus musculus
  MISC_FEATURE (20)..(20): amino acid with label
CPKPSLQQITHIANQLGLEK

SEQ ID NO: 195 - protein, 9 aa, Homo sapiens
  MISC_FEATURE (9)..(9): amino acid with label
FEALQLSLK

SEQ ID NO: 196 - protein, 12 aa, Homo sapiens
  MISC_FEATURE (12)..(12): amino acid with label
GNLENLFLQCPK

SEQ ID NO: 197 - protein, 20 aa, Homo sapiens
  MISC_FEATURE (20)..(20): amino acid with label
ITLGYTQADVGLTLGVLFGK

SEQ ID NO: 198 - protein, 21 aa, Mus musculus
  MISC_FEATURE (21)..(21): amino acid with label
VESNSEGTSSEPCADRPNAVK

SEQ ID NO: 199 - protein, 9 aa, Homo sapiens
  MISC_FEATURE (9)..(9): amino acid with label
VFSQTTICR

SEQ ID NO: 200 - protein, 10 aa, Homo sapiens
  MOD_RES (1)..(1): S-carboxyamidomethylcysteine
  MISC_FEATURE (10)..(10): amino acid with label
CLHDIALAHR

SEQ ID NO: 201 - protein, 10 aa, Mus musculus
  MOD_RES (1)..(1): S-carboxyamidomethylcysteine
  MISC_FEATURE (10)..(10): amino acid with label
CLHDIAQAHR

SEQ ID NO: 202 - protein, 12 aa, Mus musculus
  MOD_RES (1)..(1): PHOSPHORYLATION
  MISC_FEATURE (12)..(12): amino acid with label
SLPVSVPVWAFK

SEQ ID NO: 203 - protein, 12 aa, Homo sapiens
  MOD_RES (1)..(1): PHOSPHORYLATION
  MISC_FEATURE (12)..(12): amino acid with label
SLPVSVPVWGFK

SEQ ID NO: 204 - protein, 15 aa, Homo sapiens
  MISC_FEATURE (15)..(15): amino acid with label
EAEDTQVFGDLPRPR

SEQ ID NO: 205 - protein, 8 aa, Homo sapiens
  MISC_FEATURE (8)..(8): amino acid with label
GALAEAAR

SEQ ID NO: 206 - protein, 8 aa, Homo sapiens
  MOD_RES (3)..(3): PHOSPHORYLATION
  MISC_FEATURE (8)..(8): amino acid with label
LNTSDFQK

SEQ ID NO: 207 - protein, 12 aa, Homo sapiens
  MISC_FEATURE (12)..(12): amino acid with label
ILQAVNFPFLVK

SEQ ID NO: 208 - protein, 21 aa, Homo sapiens
  MOD_RES (1)..(1): PHOSPHORYLATION
  MOD_RES (3)..(3): S-carboxyamidomethylcysteine
  MISC_FEATURE (21)..(21): amino acid with label
TFCGTPDYIAPEIIAYQPYGK

SEQ ID NO: 209 - protein, 9 aa, Homo sapiens
  MISC_FEATURE (9)..(9): amino acid with label
GTEELYAIK

SEQ ID NO: 210 - protein, 10 aa, Homo sapiens
  MISC_FEATURE (10)..(10): amino acid with label
LSVEIWDWDR

SEQ ID NO: 211 - protein, 12 aa, Homo sapiens
  MISC_FEATURE (12)..(12): amino acid with label
AEFWLDLQPQAK

SEQ ID NO: 212 - protein, 20 aa, Homo sapiens
  MOD_RES (3)..(3): PHOSPHORYLATION
  MOD_RES (5)..(5): S-carboxyamidomethylcysteine
  MISC_FEATURE (20)..(20): amino acid with label
ASTFCGTPDYIAPEILQGLK

SEQ ID NO: 213 - protein, 12 aa, Homo sapiens
  MISC_FEATURE (12)..(12): amino acid with label
LLAEALNQVTQR

SEQ ID NO: 214 - protein, 12 aa, Homo sapiens
  MISC_FEATURE (12)..(12): amino acid with label
AQEALDFYGEVR

SEQ ID NO: 215 - protein, 8 aa, Homo sapiens
  MISC_FEATURE (8)..(8): amino acid with label
NNIDDVVR

SEQ ID NO: 216 - protein, 29 aa, Homo sapiens
  MOD_RES (17)..(17): PHOSPHORYLATION
  MOD_RES (21)..(21): PHOSPHORYLATION
  MISC_FEATURE (29)..(29): amino acid with label
TVEEPSNPEASSSTSVTPDVSDNEPDHYR

SEQ ID NO: 217 - protein, 24 aa, Mus musculus
  MOD_RES (2)..(2): PHOSPHORYLATION
  MISC_FEATURE (24)..(24): amino acid with label
YSDTTDSDPENEPFDEDQHSQITK

SEQ ID NO: 218 - protein, 24 aa, Homo sapiens
  MOD_RES (2)..(2): PHOSPHORYLATION
  MISC_FEATURE (24)..(24): amino acid with label
YSDTTDSDPENEPFDEDQHTQITK

SEQ ID NO: 219 - protein, 18 aa, Homo sapiens
  MOD_RES (2)..(2): PHOSPHORYLATION
  MISC_FEATURE (18)..(18): amino acid with label
DSSYYWEIEASEVMLSTR

SEQ ID NO: 220 - protein, 22 aa, Homo sapiens
  MISC_FEATURE (22)..(22): amino acid with label
EERPLFPQILSSIELLQHSLPK

SEQ ID NO: 221 - protein, 12 aa, Mus musculus
  MISC_FEATURE (12)..(12): amino acid with label
QTAQGMDYLHAK

SEQ ID NO: 222 - protein, 19 aa, Mus musculus
  MOD_RES (3)..(3): PHOSPHORYLATION
  MISC_FEATURE (19)..(19): amino acid with label
STSTPNVHMVSTTLHVDSR

SEQ ID NO: 223 - protein, 19 aa, Homo sapiens
  MOD_RES (3)..(3): PHOSPHORYLATION
  MISC_FEATURE (19)..(19): amino acid with label
STSTPNVHMVSTTLPVDSR

SEQ ID NO: 224 - protein, 13 aa, Homo sapiens
  MISC_FEATURE (13)..(13): amino acid with label
VVDPTPEQFQAFR

SEQ ID NO: 225 - protein, 23 aa, Homo sapiens
  MISC_FEATURE (23)..(23): amino acid with label
YSTPHAFTFNTSSPSSEGSLSQR

SEQ ID NO: 226 - protein, 13 aa, Homo sapiens
  MISC_FEATURE (13)..(13): amino acid with label
DSDDVPMVLVGNK

SEQ ID NO: 227 - protein, 13 aa, Homo sapiens
  MISC_FEATURE (13)..(13): amino acid with label
DSEDVPMVLVGNK

SEQ ID NO: 228 - protein, 9 aa, Homo sapiens
  MISC_FEATURE (8)..(8): amino acid with label
SFEDIHHYR

SEQ ID NO: 229 - protein, 9 aa, Homo sapiens
  MISC_FEATURE (9)..(9): amino acid with label
SFEDIHQYR

SEQ ID NO: 230 - protein, 12 aa, Homo sapiens
  MISC_FEATURE (12)..(12): amino acid with label
SYGIPYIETSAK

SEQ ID NO: 231 - protein, 12 aa, Homo sapiens
  MISC_FEATURE (12)..(12): amino acid with label
SYGIPFIETSAK

SEQ ID NO: 232 - protein, 9 aa, Homo sapiens
  MISC_FEATURE (9)..(9): amino acid with label
SFADINLYR

SEQ ID NO: 233 - protein, 11 aa, Homo sapiens
  MISC_FEATURE (11)..(11): amino acid with label
LVVVGAGGVGK

SEQ ID NO: 234 - protein, 16 aa, Homo sapiens
  MISC_FEATURE (16)..(16): amino acid with label
ALAESWNAAFLESSAK

SEQ ID NO: 235 - protein, 26 aa, Homo sapiens
  MISC_FEATURE (26)..(26): amino acid with label
SSLTIQFVEGQFVDSYDPTIENTFTK

SEQ ID NO: 236 - protein, 8 aa, Homo sapiens
  MOD_RES (3)..(3): PHOSPHORYLATION
  MISC_FEATURE (8)..(8): amino acid with label
VISYEEGK

SEQ ID NO: 237 - protein, 11 aa, Homo sapiens
  MISC_FEATURE (11)..(11): amino acid with label
VQIPIMLVGNK

SEQ ID NO: 238 - protein, 17 aa, Homo sapiens
  MOD_RES (2)..(2): S-carboxyamidomethylcysteine
  MISC_FEATURE (17)..(17): amino acid with label
GCIVDANLSVLNLVIVK

SEQ ID NO: 239 - protein, 8 aa, Homo sapiens
  MISC_FEATURE (8)..(8): amino acid with label
QGVLTHGR

SEQ ID NO: 240 - protein, 15 aa, Homo sapiens
  MISC_FEATURE (15)..(15): amino acid with label
LFPDVLFPADSEHNK

SEQ ID NO: 241 - protein, 13 aa, Homo sapiens
  MISC_FEATURE (13)..(13): amino acid with label
NIIGLLNVFTPQK

SEQ ID NO: 242 - protein, 15 aa, Homo sapiens
  MOD_RES (9)..(9): PHOSPHORYLATION
  MOD_RES (11)..(11): PHOSPHORYLATION
  MISC_FEATURE (15)..(15): amino acid with label
TAGTSFMMTPYVVTR

SEQ ID NO: 243 - protein, 8 aa, Homo sapiens
  MISC_FEATURE (8)..(8): amino acid with label
YAGYSFEK

SEQ ID NO: 244 - protein, 14 aa, Homo sapiens
  MISC_FEATURE (14)..(14): amino acid with label
ESQSHPGDFVLSVR

SEQ ID NO: 245 - protein, 12 aa, Homo sapiens
  MISC_FEATURE (12)..(12): amino acid with label
FDSLTDLVEHYK

SEQ ID NO: 246 - protein, 8 aa, Homo sapiens
  MOD_RES (4)..(4): PHOSPHORYLATION
  MISC_FEATURE (8)..(8): amino acid with label
GHEYTNIK

SEQ ID NO: 247 - protein, 9 aa, Homo sapiens
  MISC_FEATURE (9)..(9): amino acid with label
INAAEIESR

SEQ ID NO: 248 - protein, 9 aa, Homo sapiens
  MOD_RES (5)..(5): PHOSPHORYLATION
  MOD_RES (6)..(6): PHOSPHORYLATION
  MISC_FEATURE (9)..(9): amino acid with label
KGHEYTNIK

SEQ ID NO: 249 - protein, 11 aa, Homo sapiens
  MISC_FEATURE (11)..(11): amino acid with label
SNPGDFTLSVR

SEQ ID NO: 250 - protein, 12 aa, Homo sapiens
  MOD_RES (2)..(2): PHOSPHORYLATION
  MISC_FEATURE (12)..(12): amino acid with label
VYENVGLMQQQK

SEQ ID NO: 251 - protein, 11 aa, Homo sapiens
  MISC_FEATURE: amino acid with label
FLNLTENDIQK

SEQ ID NO: 252 - protein, 8 aa, Homo sapiens
  MISC_FEATURE (8)..(8): amino acid with label
LPAPSIDR

SEQ ID NO: 253 - protein, 16 aa, Homo sapiens
  MOD_RES (4)..(4): S-carboxyamidomethylcysteine
  MOD_RES (11)..(11): S-carboxyamidomethylcysteine
ALSCPGQPSNCVTIPR

SEQ ID NO: 254 - protein, 15 aa, Homo sapiens
HSEYNPQHSLLAQFR

SEQ ID NO: 255 - protein, 12 aa, Homo sapiens
  MOD_RES (5)..(5): S-carboxyamidomethylcysteine
AIENCEYAFNLK

SEQ ID NO: 256 - protein, 10 aa, Homo sapiens
  MOD_RES (7)..(7): S-carboxyamidomethylcysteine
  MISC_FEATURE (10)..(10): amino acid with label
AITTQNCNTK

SEQ ID NO: 257 - protein, 19 aa, Homo sapiens
SAGGSGGAGGGEQNGQEEK

SEQ ID NO: 258 - protein, 11 aa, Homo sapiens
WPDLHSHHELK

SEQ ID NO: 259 - protein, 10 aa, Homo sapiens
  MISC_FEATURE (10)..(10): amino acid with label
AITTQNVNTK

SEQ ID NO: 260 - protein, 13 aa, Homo sapiens
  MISC_FEATURE (13)..(13): amino acid with label
VETPVLPPVLVPR

SEQ ID NO: 261 - protein, 11 aa, Homo sapiens
  MISC_FEATURE (11)..(11): amino acid with label
DVQPVAYEEPK

SEQ ID NO: 262 - protein, 15 aa, Homo sapiens
HNEFNPQHSLLVQFR

SEQ ID NO: 263 - protein, 8 aa, Homo sapiens
GWGAEYHR

SEQ ID NO: 264 - protein, 18 aa, Homo sapiens
  MOD_RES (2)..(2): S-carboxyamidomethylcysteine
  MOD_RES (17)..(17): S-carboxyamidomethylcysteine
NCNYQHGFHPATVCCAMK

SEQ ID NO: 265 - protein, 9 aa, Homo sapiens
  MISC_FEATURE (9)..(9): amino acid with label
EYLSLGALK

SEQ ID NO: 266 - protein, 14 aa, Homo sapiens
  MISC_FEATURE (14)..(14): amino acid with label
VAELTSLSDEDSGK

SEQ ID NO: 267 - protein, 8 aa, Homo sapiens
  MISC_FEATURE (8)..(8): amino acid with label
YQCQACAR

SEQ ID NO: 268 - protein, 13 aa, Homo sapiens
  MISC_FEATURE (13)..(13): amino acid with label
AFSRPWLLQGHIR

SEQ ID NO: 269 - protein, 15 aa, Homo sapiens
  MISC_FEATURE (15)..(15): amino acid with label
DHSGSESPISDEEER

SEQ ID NO: 270 - protein, 11 aa, Homo sapiens
  MISC_FEATURE (11)..(11): amino acid with label
LSDPHAIEAEK

SEQ ID NO: 271 - protein, 10 aa, Homo sapiens
  MISC_FEATURE (10)..(10): amino acid with label
TYSTFSGLAK

SEQ ID NO: 272 - protein, 11 aa, Homo sapiens
HLDSPPAIPPR

SEQ ID NO: 273 - protein, 14 aa, Homo sapiens
ILEEAHELSEDHYK

SEQ ID NO: 274 - protein, 19 aa, Homo sapiens
TPDVFSSSPLHLQPPPLGK

SEQ ID NO: 275 - protein, 9 aa, Homo sapiens
  MISC_FEATURE (9)..(9): amino acid with label
APCQAGDLR

SEQ ID NO: 276 - protein, 20 aa, Homo sapiens
  MISC_FEATURE (20)..(20): amino acid with label
DMISMYLPGAEVPEPAAPSR

SEQ ID NO: 277 - protein, 17 aa, Homo sapiens
  MISC_FEATURE (17)..(17): amino acid with label
SEASSSPPVVTSSSHSR

SEQ ID NO: 278 - protein, 10 aa, Homo sapiens
  MOD_RES (7)..(7): PHOSPHORYLATION
  MISC_FEATURE (10)..(10): amino acid with label
LIEDNEYTAR

SEQ ID NO: 279 - protein, 9 aa, Homo sapiens
  MISC_FEATURE (9)..(9): amino acid with label
LLLNAENPR

SEQ ID NO: 280 - protein, 17 aa, Homo sapiens
  MOD_RES (5)..(5): S-carboxyamidomethylcysteine
  MISC_FEATURE (17)..(17): amino acid with label
LTTVCPTSKPQTQGLAK

SEQ ID NO: 281 - protein, 15 aa, Homo sapiens
  MISC_FEATURE (15)..(15): amino acid with label
TQFNSLQQLVAYYSK

SEQ ID NO: 282 - protein, 18 aa, Mus musculus
  MOD_RES (16)..(16): PHOSPHORYLATION
  MISC_FEATURE (18)..(18): amino acid with label
EAPEPMELDDPKRTGYIK

SEQ ID NO: 283 - protein, 18 aa, Homo sapiens
  MOD_RES (16)..(16): PHOSPHORYLATION
  MISC_FEATURE (18)..(18): amino acid with label
EAPEPMELDGPKGTGYIK

SEQ ID NO: 284 - protein, 12 aa, Homo sapiens
  MISC_FEATURE (12)..(12): amino acid with label
ELSAVTFPDIIR

SEQ ID NO: 285 - protein, 14 aa, Homo sapiens
  MISC_FEATURE (14)..(14): amino acid with label
FHDLLSQLDDQYSR

SEQ ID NO: 286 - protein, 20 aa, Homo sapiens
  MOD_RES (11)..(11): PHOSPHORYLATION
  MISC_FEATURE (20)..(20): amino acid with label
LQTTDNLLPMSPEEFDEVSR

SEQ ID NO: 287 - protein, 9 aa, Homo sapiens
  MISC_FEATURE (9)..(9): amino acid with label
QYLAQWLEK

SEQ ID NO: 288 - protein, 13 aa, Homo sapiens
  MISC_FEATURE (13)..(13): amino acid with label
TELISVSEVHPSR

SEQ ID NO: 289 - protein, 13 aa, Homo sapiens
  MISC_FEATURE (13)..(13): amino acid with label
VMAAENIPENPLK

SEQ ID NO: 290 - protein, 9 aa, Homo sapiens
  MISC_FEATURE (9)..(9): amino acid with label
FPELNYQLK

SEQ ID NO: 291 - protein, 13 aa, Homo sapiens
  MISC_FEATURE (13)..(13): amino acid with label
GLSIEQLTTLAEK

SEQ ID NO: 292 - protein, 11 aa, Homo sapiens
  MISC_FEATURE (11)..(11): amino acid with label
TQIQSVEPYTK

SEQ ID NO: 293 - protein, 22 aa, Homo sapiens
  MOD_RES (2)..(2): S-carboxyamidomethylcysteine
  MOD_RES (20)..(20): PHOSPHORYLATION
  MISC_FEATURE (22)..(22): amino acid with label
YCRPESQEHPEADPGSAAPYLK

SEQ ID NO: 294 - protein, 11 aa, Homo sapiens
  MOD_RES (5)..(5): PHOSPHORYLATION
  MISC_FEATURE (11)..(11): amino acid with label
AVDGYVKPQIK

SEQ ID NO: 295 - protein, 12 aa, Homo sapiens
  MISC_FEATURE (12)..(12): amino acid with label
LSPPAGLFTSAR

SEQ ID NO: 296 - protein, 11 aa, Homo sapiens
  MISC_FEATURE (11)..(11): amino acid with label
NLWNLKPFTTR

SEQ ID NO: 297 - protein, 8 aa, Homo sapiens
  MISC_FEATURE (8)..(8): amino acid with label
YYTPVLAK

SEQ ID NO: 298 - protein, 10 aa, Homo sapiens
  MOD_RES (8)..(8): PHOSPHORYLATION
  MOD_RES (9)..(9): PHOSPHORYLATION
  MISC_FEATURE (10)..(10): amino acid with label
ALRADENYYK

SEQ ID NO: 299 - protein, 23 aa, Homo sapiens
  MOD_RES (12)..(12): PHOSPHORYLATION
  MOD_RES (14)..(14): PHOSPHORYLATION
  MISC_FEATURE (23)..(23): amino acid with label
EALPMDTEVYESPYADPEEIRPK

SEQ ID NO: 300 - protein, 23 aa, Homo sapiens
  MOD_RES (14)..(14): PHOSPHORYLATION
  MISC_FEATURE (23)..(23): amino acid with label
EALPMDTEVYESPYADPEEIRPK

SEQ ID NO: 301 - protein, 9 aa, Homo sapiens
  MISC_FEATURE (9)..(9): amino acid with label
LIATTAHEK

SEQ ID NO: 302 - protein, 9 aa, Homo sapiens
  MOD_RES (3)..(3): S-carboxyamidomethylcysteine
  MOD_RES (7)..(7): S-carboxyamidomethylcysteine
  MISC_FEATURE (9)..(9): amino acid with label
MGCPAGCPR

SEQ ID NO: 303 - protein, 23 aa, Homo sapiens
QTWNLQGQALEQAIISQKPQLEK

SEQ ID NO: 304 - protein, 12 aa, Homo sapiens
  MISC_FEATURE (12)..(12): amino acid with label
FTTASIGLPPPR

SEQ ID NO: 305 - protein, 7 aa, Homo sapiens
  MOD_RES (5)..(5): PHOSPHORYLATION
  MISC_FEATURE (7)..(7): amino acid with label
GQEVYVK

SEQ ID NO: 306 - protein, 13 aa, Homo sapiens
  MISC_FEATURE (13)..(13): amino acid with label
YIGGNLFTSAFTR

SEQ ID NO: 307 - protein, 12 aa, Mus musculus
  MISC_FEATURE (12)..(12): amino acid with label
AAALEEHNAAMK

SEQ ID NO: 308 - protein, 9 aa, Homo sapiens
  MOD_RES (5)..(5): PHOSPHORYLATION
  MISC_FEATURE (9)..(9): amino acid with label
GGFDSPFYR

SEQ ID NO: 309 - protein, 22 aa, Mus musculus
  MOD_RES (6)..(6): PHOSPHORYLATION
  MISC_FEATURE (22)..(22): amino acid with label
GVSFGSGQLPPYDHLFEVALPK

SEQ ID NO: 310 - protein, 12 aa, Homo sapiens
  MISC_FEATURE (12)..(12): amino acid with label
LSQAPLLPSLLK

SEQ ID NO: 311 - protein, 14 aa, Homo sapiens
  MOD_RES (2)..(2): S-carboxyamidomethylcysteine
  MISC_FEATURE (14)..(14): amino acid with label
VCHTELLLSQVSQK

SEQ ID NO: 312 - protein, 21 aa, Homo sapiens
  MOD_RES (5)..(5): PHOSPHORYLATION
  MISC_FEATURE (21)..(21): amino acid with label
VGFGSGQPPPYDHLFEVALPK

SEQ ID NO: 313 - protein, 10 aa, Homo sapiens
  MOD_RES (1)..(1): PHOSPHORYLATION
  MISC_FEATURE (10)..(10): amino acid with label
SLHAEELVGR

SEQ ID NO: 314 - protein, 10 aa, Homo sapiens
  MOD_RES (1)..(1): PHOSPHORYLATION
  MISC_FEATURE (10)..(10): amino acid with label
YTEFLTGLGR

SEQ ID NO: 315 - protein, 23 aa, Mus musculus
  MOD_RES (3)..(3): PHOSPHORYLATION
  MISC_FEATURE (23)..(23): amino acid with label
AISSEGARPAVDLSFQPSQPLSK

SEQ ID NO: 316 - protein, 13 aa, Homo sapiens
  MISC_FEATURE (13)..(13): amino acid with label
GQPEGPLPSSSPR

SEQ ID NO: 317 - protein, 11 aa, Homo sapiens
  MOD_RES (3)..(3): PHOSPHORYLATION
  MISC_FEATURE (11)..(11): amino acid with label
GYTISDSAPSR

SEQ ID NO: 318 - protein, 15 aa, Mus musculus
  MISC_FEATURE (15)..(15): amino acid with label
ITVPPEGPLPSSSPR

SEQ ID NO: 319 - protein, 25 aa, Mus musculus
  MISC_FEATURE (25)..(25): amino acid with label
SLLGLDSGDLQGGSDSSSDPSTHVR

SEQ ID NO: 320 - protein, 25 aa, Homo sapiens
  MISC_FEATURE (25)..(25): amino acid with label
SLLGLDSGELQSGPESSSSPGVHVR

SEQ ID NO: 321 - protein, 9 aa, Homo sapiens
  MISC_FEATURE (9)..(9): amino acid with label
IIPTLPSDK

SEQ ID NO: 322 - protein, 9 aa, Homo sapiens
  MISC_FEATURE (9)..(9): amino acid with label
LSYAFSVWR

SEQ ID NO: 323 - protein, 12 aa, Homo sapiens
  MISC_FEATURE (12)..(12): amino acid with label
TQSLNEAFAALR

SEQ ID NO: 324 - protein, 17 aa, Homo sapiens
  MISC_FEATURE (17)..(17): amino acid with label
YIDFLYQVLQSDELDSK

SEQ ID NO: 325 - protein, 10 aa, Homo sapiens
  MISC_FEATURE (10)..(10): amino acid with label
LGDLYEEEMR

SEQ ID NO: 326 - protein, 19 aa, Homo sapiens
  MISC_FEATURE (19)..(19): amino acid with label
LLQDSVDFSLADAINTEFK

SEQ ID NO: 327 - protein, 12 aa, Homo sapiens
  MISC_FEATURE (12)..(12): amino acid with label
FDTLWQLVEYLK

SEQ ID NO: 328 - protein, 28 aa, Homo sapiens
  MOD_RES (17)..(17): PHOSPHORYLATION
  MOD_RES (21)..(21): PHOSPHORYLATION
  MISC_FEATURE (28)..(28)
ITSPDKPRPMPMDTSVYESPYSDPEELK

SEQ ID NO: 329 - protein, 28 aa, Homo sapiens
  MOD_RES (21)..(21): PHOSPHORYLATION
  MISC_FEATURE (28)..(28): amino acid with label
ITSPDKPRPMPMDTSVYESPYSDPEELK

SEQ ID NO: 330 - protein, 14 aa, Homo sapiens
  MISC_FEATURE (14)..(14): amino acid with label
SLGGYVLSLVHDVR

SEQ ID NO: 331 - protein, 11 aa, Homo sapiens
  MISC_FEATURE (11)..(11): amino acid with label
TVYHYLISQDK

SEQ ID NO: 332 - protein, 16 aa, Mus musculus
  MISC_FEATURE (16)..(16): amino acid with label
AYYALNAQPSAEELSK

SEQ ID NO: 333 - protein, 16 aa, Homo sapiens
  MISC_FEATURE (16)..(16): amino acid with label
AYYALNAQPSTEELSK

SEQ ID NO: 334 - protein, 21 aa, Homo sapiens
  MISC_FEATURE (21)..(21): amino acid with label
DSTEQEEAGPEILSNEHVGAR

SEQ ID NO: 335 - protein, 14 aa, Mus musculus
  MISC_FEATURE (14)..(14): amino acid with label
IADSVNLPLDGVLK

SEQ ID NO: 336 - protein, 16 aa, Homo sapiens
  MISC_FEATURE (16)..(16): amino acid with label
IENKPLQEQLSVNQIK

SEQ ID NO: 337 - protein, 17 aa, Homo sapiens
  MISC_FEATURE (17)..(17): amino acid with label
MTNSPVLPVGSTTNGSR

SEQ ID NO: 338 - protein, 19 aa, Mus musculus
  MISC_FEATURE (19)..(19): amino acid with label
TDGTVEVGAAQQAGSLEQK

SEQ ID NO: 339 - protein, 9 aa, Homo sapiens
  MISC_FEATURE (9)..(9): amino acid with label
VAVDGNVIR

SEQ ID NO: 340 - protein, 13 aa, Homo sapiens
  MISC_FEATURE (13)..(13): amino acid with label
DGHAVSIEEYLQR

SEQ ID NO: 341 - protein, 15 aa, Homo sapiens
  MISC_FEATURE (15)..(15): amino acid with label
SNTPSPLNLSSTSSK

SEQ ID NO: 342 - protein, 10 aa, Homo sapiens
  MISC_FEATURE (10)..(10): amino acid with label
TEPLDFNDYK

SEQ ID NO: 343 - protein, 11 aa, Homo sapiens
  MISC_FEATURE (11)..(11): amino acid with label
VLQIVDNTVSR

(19) United States
(12) Patent Application Publication
(10) Pub. No.: US 2017/0370942 A1
(43) Pub. Date: Dec. 28, 2017

(54) HIGHLY MULTIPLEXED ABSOLUTE QUANTIFICATION OF MOLECULES ON THE SINGLE CELL LEVEL

(71) Applicants: UNIVERSITAT ZURICH, Zurich (CH); ETH ZURICH, Zurich (CH)

(72) Inventors: Paola PICOTTI, Zurich (CH); Andrea JACOBS, Zurich (CH); Serena DI PALMA, Zurich (CH); Bernd BODENMILLER, Zurich (CH)

(73) Assignees: UNIVERSITAT ZURICH, Zurich (CH); ETH ZURICH, Zurich (CH)

(21) Appl. No.: 15/503,727

(22) PCT Filed: Aug. 14, 2015
(86) PCT No.: PCT/EP2015/068797

§ 371 Date: Feb. 14, 2017

(30) Foreign Application Priority Data
Aug. 14, 2014 (EP) 14181092.9

(51) Int. Cl. G01N 33/68

(52) U.S. Cl. CPC G01N 33/6848; G01N 2560/00

(57) ABSTRACT

The invention relates to a method for determining a biomarker on single cell level by counting a first portion of a cell sample, subjecting said first portion to conditions whereby the biomarker is fragmented, adding to a known number n(k) of labelled biomarker fragments, measuring a first and a second parameter of said first portion, wherein said first parameter corresponds to the amount of said biomarker fragment and said second parameter corresponds to the amount of said labelled biomarker fragment yielding a biomarker fragment value, v(u), and a labelled biomarker fragment value v(k), respectively, and relating v(u) and v(k) with n(k) and c(1), thereby determining an average number of biomarker molecules per cell, m(u), of said first portion. Subsequently, a second portion of the cell sample is contacted with a label specific for the biomarker, a value of the label is determined for the second portion, yielding a single cell measurement value s(u) for the cells of the second portion, a mean measurement value m(s) is determined, and a number of biomarker molecules, n(u) is computed from s(u), m(s) and m(u) for each cell.

### BACKGROUND OF THE INVENTION

Elemental mass spectrometry-based flow cytometry (mass cytometry) is a method to characterize single cells or particles via the replacement of fluorochrome-labeled binding reagents with elemental metal isotope-labelled binding reagents (i.e. antibodies, aptamers, DARPINs, chemical linkers, or other affinity reagents). Because there are many stable metal isotopes available (˜100), and no or very little overlap between measurement channels is observed in mass cytometry measurements, dozens of molecules (parameters) can be measured as readily as one. The mass cytometer used to read the metal tags is an inductively-coupled plasma mass spectrometer (ICP-MS), which in its current configuration allows analyzing simultaneously up to 135 isotopes and therefore molecules (Bandura et al. Anal Chem. 2009 Aug. 15; 81(16):6813-22).

In a typical workflow (similar to fluorescence based cytometry), cells are first incubated with affinity binders conjugated to pure isotopes and subsequently the cell suspension is injected as a single cell stream into the mass cytometer. Single cell droplets are generated via nebulization and are carried by an argon gas stream into a ˜7500 degrees Kelvin plasma where each single cell is completely atomized and ionized. Thereby generated metal ions are then directed into a time-of-flight (TOF) mass spectrometer and the mass over charge ratio and number of metal ions is measured per cell, thereby facilitating determination the qualitative abundance of the target epitope/molecules. Similarly, other single cell technologies exist to characterize single cells using binding reagents; these include flow cytometry, immunocytochemical and immunohistochemical methods coupled to microscopy and other optical devices, e.g. implemented in microfluidic devices.

The value of affinity binder based single cell analysis technologies, including mass cytometry (which includes the routine measurement of up to 42 parameters with the potential to measure more than 100), has extensively been documented (Bendall et al. Science. 2011 May 6; 332(6030):687-96; Giesen et al. Nat Methods. 2014 April; 11(4):417-22; Bodenmiller et al. Nat Biotechnol. 2012 September; 30(9):858-67; Bjornson et al. Curr Opin Immunol. 2013 August; 25(4):484-94). Typically, these single cell analysis methods are used to study cellular phenotypes. These phenotypes include the expression of biomarkers, e.g. proteins and their modifications, and phenotypes that are based on the visualization of cell shape, size and spatial distribution of biomarkers. All of these biomarkers have in common that they allow drawing conclusions about cell type, cell behavior and cell response. The analysis of such biomarkers is extensively used in basic research, pharmaceutical and biomedical research and in clinical practice. In the context of pharmaceutical research these biomarkers are e.g. used to identify appropriate drugs for a given cell type and disease, and for clinical/patient samples, the analysis of the biomarkers can be prognostic, predictive and diagnostic for treatment and disease progression, and thus are essential for the patient management.

An essential feature to describe cellular biomarkers and thus phenotypes is the absolute number of molecules per single cell. The knowledge about the numbers of biomarker-molecules per single cell is essential for a systematic and automated classification of patients. Currently, patients are subjectively classified by pathologists based on qualitative immunohistochemical tissue analysis, leading to known high error rates in classification. Absolute copy number on the other hand would enable algorithm based, objective patient classification. Absolute molecule numbers per single cell are also essential to compute many cellular properties, including kinetic rates of enzymatic reactions, the stoichiometry of protein modifications, thresholds of cellular processes and network structures. These cellular properties are highly relevant for drug development and for understanding the basic mechanisms of life. In addition, only absolute molecule numbers make single cell data comparable between experiments and laboratories. As such, there is a significant need for methods and standards that overcome the lack of absolute quantification on the single cell level. No method exists to accurately quantify the molecule number of a given biomarker via binding reagents on the single cell level, especially if multiple biomarkers or protein modifications need to be quantified absolutely and simultaneously.

In “quantitative” flow cytometry the fluorescence intensity measured on single cells of interest, which is derived by the bound fluorescently labelled affinity binders, is compared to a calibration standard, typically beads with a known number of bound antibody. Using beads with different numbers of bound antibodies, a calibration curve relating mean fluorescence intensity into a “molecule number” per cell can be drawn. However, what in this case is truly determined is the number of bound antibodies with a fluorophore to a given cell, but not the number of the target molecules. This is based on the fact that antibodies, as well as all affinity binders, do not bind all, but only the accessible epitopes. Furthermore antibodies show unspecific background binding on cells, and the background binding can be specific of a given cell type, that is difficult to mimic on beads. Antibodies have two binding sites, of which it is unknown if both or just one is occupied. Ultimately, any protocol to label affinity binders with a reporter will generate active, but unlabeled binders. Thus “quantitative flow cytometry (qFACS)” does not allow determining absolute numbers of molecules per cell.

In another quantitative single cell analysis approach cellular molecules are labelled with a fluorescent protein (or other reporters that can be detected in single cells) to determine the number of the fluorescent protein molecules by using appropriate standards by microscopy and flow cytometry (Newman et al. Nature 441, 2006, 840-846). However, using this approach it is not possible to quantify protein modifications and even more important, samples in which no fluorescent protein can be introduced, e.g. all human donor and patient, samples cannot be quantified. Furthermore, these approaches typically do not allow for higher multiplexed quantifications, but only of one or few molecules simultaneously.

The problem underlying the present invention is to provide the means for determining the number of molecules of a single or multiple biomarkers in single cells in a plurality of cells. This problem is solved by the subject-matter of the independent claims.

### TERMS AND DEFINITIONS

Amino acid sequences are given from amino to carboxyl terminus.

In the context of the present specification, the term biomarker refers to natural or synthetic molecules that are present in or on cells and can be quantified by single cell analysis methods. Non-limiting examples for biomarkers are proteins, protein modifications such as protein phosphorylation or acetylation, modified proteins exemplified by but not limited to phosphorylated or acetylated proteins, peptides, RNA, DNA, small molecule compounds, metabolites, mono- and polysaccharides and metalorganic compounds.

In the context of the present specification, the term relative quantity refers to a quantity that is expressed in relation to another value. Examples for relative quantities would be ratios or percentages. In contrast, absolute quantities give an absolute occurrence value or in other words it tells how many or much occurrences are present. An example for an absolute quantity would be the number of molecules per cell.

In the context of the present specification, the term affinity binder refers to any molecule that binds to specific target molecules through molecular interactions (e.g. hydrogen bonding, Van der Waals forces, electrostatic forces, hydrophobic forces, etc.). Non-limiting examples for affinity binders include antibodies and parts thereof, DARPINs (designed ankyrin repeat proteins), RNA or DNA-based affinity binders such as aptamers, etc. Affinity binders can be labelled with a wide range of reporters including fluorophores, an atom of an element with a defined isotope distribution, a compound with a defined isotope mixture, a catalytically active moiety, a nucleic acid, molecules with a defined mass over charge ratio (m/z) etc. In some cases, affinity based binding may lead to covalent linkage. One example are active site-specific inhibitors that covalently link to their target.

In the context of the present specification, the term aptamer refers to single-stranded DNA or RNA (ssDNA or ssRNA) molecules, such as, by way of non-limiting example, RNA aptamers or L-RNA aptamers (see U.S. Pat. No. 6,605,713 and documents citing this publication, all of which are incorporated herein by reference), that can bind to pre-selected targets including proteins and peptides with high affinity and specificity.

In the context of the present specification, the term Designed Ankyrin Repeat Proteins (DARPins) refers to are engineered antibody mimetic proteins typically exhibiting highly specific and high-affinity target protein binding (see US2012142611 and documents citing this application, all of which are incorporated herein by reference). They are derived from naturally occurring ankyrin proteins, a protein class that is mediating high-affinity protein-protein interactions in nature.

In the context of the present specification, the term antibody is used in its meaning known in the art of cell biology and immunology; it refers to whole antibodies including but not limited to immunoglobulin type G (IgG), type A (IgA), type E (IgE) or type M (IgM), any antigen binding fragment (for example, derived of an IgG molecule) or single chains thereof and related or derived constructs. A whole antibody is a glycoprotein comprising at least two heavy (H) chains and two light (L) chains inter-connected by disulfide bonds. Each heavy chain is comprised of a heavy chain variable region (V_{H}) and a heavy chain constant region (C_{H}). Each light chain is comprised of a light chain variable region (abbreviated herein as V_{L}) and a light chain constant region (C_{L}). The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component of the classical complement system.

In the context of the present specification, the term metalorganic compound refers to organic compounds containing a metal atom that is directly bound to a carbon atom.

According to a first aspect of the invention, a method for determining the number of molecules of a biomarker on a single cell level is provided. The method comprises the following steps:
A first portion of a cell population from a mammalian, particularly human, cell sample particularly taken from a previously prepared cell culture or directly from a patient, is obtained. The cell population is characterized by the presence of the biomarker.
A cell number, c(1), of the first portion is obtained by counting the cells,
An average number of biomarker molecules per cell of the first portion of cells is determined, by conducting the following steps:
   i. In a fragmentation step, the cells of the first portion are subjected to conditions whereby the biomarker is fragmented, yielding a biomarker fragment.
   ii. To these biomarker fragments a known number of labelled biomarker fragment molecules, n(k), is added. The labelled biomarker fragment molecules are characterized in that they differ from the biomarker fragments only in a detectable label (e.g., an isotope label).
   iii. A first and a second parameter of the first portion is measured. The first parameter corresponds to the amount of the biomarker fragment and the second parameter corresponds to the amount of the labelled biomarker fragment. This measurement yields a biomarker fragment value, v(u), and a labelled biomarker fragment value v(k), respectively. A non-limiting example of measured parameter is mass spectroscopy signal intensity, another fluorescence intensity.
   iv. In a first computational step the biomarker fragment value, v(u), the labelled biomarker fragment value v(k), the number of labelled biomarker fragments n(k) and the number of cells of the first portion c(1) are related. An average number of biomarker molecules per cell, m(u), of the first portion of cells is determined thereby.

The above procedure (determination of cell number, steps i, ii, iii, iv) is repeated with further cell populations that show different levels of v(u). These can be different cell types and lines, or an identical cell line in which v(u) was artificially overexpressed, or its levels reduced. These average numbers of biomarkers of different levels per cell are used to generate a calibration curve.

The relative quantity of the biomarker on the single cell level is determined and related to the average number of biomarker molecules per cell by the following steps:
v. The cells of a second portion of the cell population subject to analysis are first labelled with a marker that allows to distinguish the cells which resulted in a given average copy number per cell. These cells are then subjected to conditions allowing a labelled affinity binder to bind specifically to said biomarker.
vi. A value of the bound labelled affinity binder (by non-limiting example, a fluorescence intensity of a fluorescence label specifically attached to the affinity binder) is measured for a plurality of the cells of the second portions. The value thus determined corresponds to the amount of the biomarker in the cell, yielding a single cell measurement value, s(u), for a plurality of cells of the second portion of cells. From these single cell measurement values, a mean measurement value, m(s)1 . . . n, for the plurality of cells is determined.
vii. In a second computational step, the mean measurement values m(s)1 . . . n and the average number of biomarker molecules per cell, m(u)1 . . . n are related and a calibration curve is computed. This can be achieved using standard regression methods. Should no linear behaviour be observed, curve fitting can be employed.
viii. In a third computational step, the single cell measurement value s(u), is related to the calibration curve, thereby yielding a number of biomarker molecules per single cell, n(u).

According to an alternative to this first aspect of the invention, the method for determining the number of molecules of a biomarker on a single cell level comprises the following steps:
A first portion of a cell population from a mammal, human or human patient is obtained and a cell number, c(1), of the first portion is obtained by counting the cells.
An average number of biomarker molecules per cell of the first portion of cells is determined, by conducting the steps I to iv as indicated for the previously outlined alternative of this aspect of the invention.
The relative quantity of the biomarker on the single cell level is determined and related to the average number of biomarker molecules per cell by the following steps:
   i. The cells of the second portion are subjected to conditions allowing a labelled affinity binder to bind specifically to said biomarker.
   ii. A value of the bound labelled affinity binder is measured for a plurality of the cells of the second portion. The value thus determined corresponds to the amount of the biomarker in the (each) cell, yielding a single cell measurement value, s(u), for a plurality of cells of the second portion of cells. From these single cell measurement values a mean measurement value, m(s), for the plurality of cells is determined.
   iii. In a second computation step, the single cell measurement value s(u), the mean measurement value m(s) and the average number of biomarker molecules per cell, m(u) are related, thereby yielding a number of biomarker molecules per single cell, n(u).

In certain embodiments the first computational step is calculated using formula I:

$$\frac{\frac{v ( u )}{v ( k )} * n ( k )}{c ( 1 )} = m ( u )$$

In certain embodiments the second computational step is calculated using linear or robust regression methods.

In certain embodiments the third computation step is calculated using y=ax+b.

In certain embodiments the single cell analysis technology has a detection limit, in which the term b (x=0) is the lowest detectable copy number of a molecule, yielding y=b. (see Skoog et al., Principles of Instrumental Analysis, 6^{th} edition, Cengage Learning (2006); ISBN 978-0495012016). Alternatively, d(s) can be computed based on the measurement of a single cell sample if the detection limit of the instrument and the average signal expected per affinity binder is known.

In certain embodiments the ratio between s(u)1 . . . n and m(u)1 . . . n in the second computation step is not linear. It is described by a response function (calibration curve) defined by the instrument response (and/or antibody binding behaviour) over the measured dynamic range.

In certain embodiments, the plurality of cells of step vi. above is a number statistically representative of analysed cell population. In certain embodiments, the plurality of cells of step vi. above is >1000, >5000, or >10.000.

In certain embodiments of any aspect of the invention the biomarker is a protein, a peptide derived from a protein, a peptide, a small molecule compound, DNA, RNA, mono-saccharide, poly-saccharide or metalo-organic compound.

In certain embodiments of any aspect of the invention the biomarker is a post-translationally modified peptide derived from a protein.

In certain embodiments of any aspect of the invention the amino acid sequence of the biomarker fragment is selected from the second column in the table shown in FIG. 4.

In certain embodiments the method used in the fragmentation step is enzymatic digestion.

In certain embodiments, the (biomarker) fragments resulting from the fragmentation step, e.g. peptides, are subjected to mass spectrometry (MS), particularly to MS-based methods including but not limited to collision induced dissociation, infrared multiphoton dissociation, blackbody infrared radiative dissociation, electron-capture dissociation, negative electron-transfer dissociation, electron-detachment dissociation or surface induced dissociation in step iii. See also: Chhabil Dass, Fundamentals of Contemporary Mass Spectrometry, ISBN: 978-0-471-68229-5; Paizs and Suhai, Mass Spectrum Rev. 2005 July-August; 24(4):508-48; Mass Spectrometry of Proteins and Peptides: Methods and Protocols, Second Edition (Methods in Molecular Biology) 2nd ed. 2009, ISBN-13: 978-1934115480; Stingl et al., Biochim Biophys Acta. 2006 December; 1764(12):1842-52. Epub 2006 Oct. 3.

In certain embodiments of any aspect of the invention, the biomarker fragment is labelled with a stable marker isotope, yielding an isotopically labeled biomarker fragment. The isotopically labeled biomarker fragment is synthetically formed to have incorporated therein at a selected position a stable marker isotope of an element, wherein the element exists at the selected position in the naturally occurring biomarker molecule. The stable marker isotope is an isotope which has a mass that is different from the mass of the most abundantly occurring isotope of the element in nature, such that the isotopically labeled biomarker fragment has a molecular weight different from the molecular weight of the naturally occurring biomarker molecule. In certain embodiments the biomarker is a peptide; in this case amino acids labeled with stable isotopes that distinguish them from naturally occurring amino acids may be used for the peptide synthesis of the labelled biomarker fragment.

In certain embodiments the first and second parameter that corresponds to the amount of biomarker or labelled biomarker is selected MS1 mass spectrometry signal intensity values at a given m/z value, or MS2 peptide fragment intensity values at a single or multiple m/z value, or MS3 peptide fragment intensity values at a single or multiple m/z value.

In certain embodiments the value of the bound labelled affinity binder that corresponds to the amount of biomarker molecules is selected from fluorescence intensity value, mass spectrometry signal intensity value, spectrophotometric values, western blot intensity values, RNA sequencing values and quantitative PCR values.

In certain embodiments of any aspect of the invention the labelled affinity binder is selected from an antibody, RNA/DNA binders including aptamers, DARPINs (designed ankyrin repeat proteins).

In certain embodiments the labelled affinity binder is labelled with fluorophores, stable isotopes, DNA or RNA (Sano et al. 1992, Science 258:120-122; Niemeyer et al., 2007, Nat Protoc 2:1918-1930; Fredriksson et al., 2007 Nat. Methods 4:327-329).

In certain embodiments the number of biomarker molecules is determined for a plurality of different biomarkers. In certain embodiments the plurality of biomarkers comprises 1, 2, 3, 4, 5, 6, 7, 8, 9, 10, 10-20, 20-30, 30-50, 50-70, 50-100, or 50-150, 50-250 and 100-500 different biomarkers.

In certain embodiments the first labelled biomarker fragment behaves similarly to said first (unlabeled) biomarker fragment with respect to a separation method, and said first labelled biomarker fragment behaves differently to said first (unlabeled) biomarker fragment with respect to a detection method. One non-limiting example of a separation method is liquid-chromatography. An example for a detection method is mass spectrometry.

In certain embodiments of any aspect of the invention the average number of biomarker molecules of interest per cell, m(u), is determined in a portion of standard cells, wherein the biomarker of interest is present and detectable in the single cell analysis. Other portions of the standard cells, that contain a unique label to identify them in the single cell measurement (e.g. metal label via small molecule or antibody, fluorophore label for immunofluorescence analysis, etc.) are then added to the cell portion of interest and the single cell measurement value s(u) and the mean measurement value m(s) are determined for the cell portion of interest and the added standard cells.

In certain embodiments of any aspect of the invention several standard cells are used that express m(u) at different levels. Relating the measured average number of biomarker molecules of all standard cells m(u)1 . . . n to m(s)1 . . . n enables to compute a calibration curve. Values of the cells of interest s(u) yields the number of biomarker molecules per single cell of interest, n(u), via the equation derived from the calibration curve.

The standard cells are thus characterized in that the biomarker is present and detectable in both step iii (measurement of portion 1) and step vi (single cell analysis).

For single cell analysis, the standard cells are characterized by a unique label (by non-limiting example, with a metal for mass cytometry) that identifies them as standard cells.

If several sets of standard cells are used to generated a calibration curve, each standard cell type is characterized by a unique label (by non-limiting example, with a metal for mass cytometry) that identifies them as standard cells.

According to an alternative aspect of the invention a method for quantifying a biomarker on a single cell level is provided, comprising the steps of:
a) Providing a cell population comprising the single cells, wherein the single cells comprise the biomarker,
b) Taking at least a first and a second portion of the cell population and obtaining the cell number of each portion, or at least the first portion,
c) Obtaining an average number of biomarker molecules per cell by
   i. adding a known number of molecules of a labelled biomarker (or fragments thereof) to the first portion,
   ii. obtaining a relative quantity value for the biomarker (or fragment thereof) and the labelled biomarker (or fragment thereof) in the first portion, and
   iii. relating the relative quantity of the biomarker (or fragment thereof) and the labelled biomarker (or fragment thereof) with the known number of molecules of the labelled biomarker (or fragment thereof) and the number of cells of the first portion yielding an average number of biomarker molecules per cell in the first portion;
d) determining the relative quantity of the biomarker on the single cell level and relate it to the average number of biomarker molecules per cell, comprising the steps of
   i. Subjecting the cells of the second portion to conditions whereby a labelled affinity binder specifically binds to the biomarker,
   ii. Measuring a value of the bound labelled affinity binder for single cells, wherein the value corresponds to the amount of the biomarker in/on the single cells, yielding a single measurement value for a plurality of cells of the second portion, and determining from the single measurement value a mean measurement value for the plurality of cells, and
   iii. Relating the single measurement value, the mean measurement value and the average number of biomarker molecules per cell, yielding a number of biomarker molecules in the single cell.

As related above, a calibration curve may similarly be created from a number of measurements of cell populations expressing the biomarker at different levels.

In certain embodiments, step c) is repeated several times with further cell populations that show different levels of v(u). As before, the cells used in these subsequent steps for calibration purposes can be different cell types and lines, or an identical cell line in which v(u) was artificially overexpressed, or its levels reduced. These average numbers of biomarkers of different levels per cell are used to generate the calibration curve.

In certain embodiments the cell population comprises single prokaryotic, eukaryotic, mammalian, human or human cancer cells characterized by the presence of the biomarker.

In certain embodiments, the cell population is part of a tissue such as a tumor and the single cell analysis technology analyses a tissue section derived from the tumor. For such analysis, standard cells are identically processed as the tissue section, e.g. processed with formalin fixation and paraffin embedding or embedded in a matrix and then frozen akin to frozen tissue analysis. Then a section of the standard cells and tissue is cut with equal thickness and the BOI are labelled with the affinity binders. As above, the copy number per single cell is computed.

In certain embodiments, a tissue section is generated. In this case, the skilled artisan will recognize that often not entire cells are subjected to the method of the invention, but at least for a subpopulation of the cells, only parts of a cell are measured. The method of the invention will nonetheless deliver useful results, as either serial sections can be measured to determine the whole cell volume, or the proportion of cells that are cut can be estimated.

In certain embodiments, the tissue section does not comprise a whole cell, but parts of a cell. To determine copy numbers per cell either serial sections can be analysed to reconstruct the whole cell volume, or the fraction of cell is estimated to calculate the copy number per cell.

In certain embodiments, cells are first lysed followed by fragmentation of the proteins by enzymes into peptides. The resulting peptides are then once more fragmented during detection by mass spectroscopy to determine the amino acid sequence.

Wherever alternatives for single separable features such as, for example, a label for an affinity binder or coding sequence of a biomarker fragment are laid out herein as “embodiments”, it is to be understood that such alternatives may be combined freely to form discrete embodiments of the invention disclosed herein.

The invention is further illustrated by the following examples and figures, from which further embodiments and advantages can be drawn. These examples are meant to illustrate the invention but not to limit its scope.

### BRIEF DESCRIPTION OF THE FIGURES

FIG. 1 shows the approach for absolute quantification of the number of biomarker molecules on the single cell level for single population (A) and multiple population (calibration curve, B) relation building of copy number and mean cytometry intensity values.

FIG. 2 shows 2 shows a generalized workflow for an assay for absolute quantification of biomarkers of interest (BOI) on the single cell level.

FIG. 3: shows example calibration curves for CD44 (left), vimentin (middle) and c-Met (right) X-axis: mean cytometry intensity values; Y-axis: copy number determined by mass spec; different colour/data point shapes correspond to different cell populations; multiple data points of similar colour/data point shapes correspond to data replicates.

FIG. 4 shows an example for an assay to quantify the number of Her2 marker molecules in cells.

FIG. 5 is a list of representative peptide fragments characteristic of biomarkers.

### EXAMPLES

The invention discloses a general approach to develop assays that allow to absolutely quantify biomarkers on the single cell level. This approach uses cell population based techniques such as protein mass spectrometry to generate cell standards, which can be the cells studied or “standard cells” that express the biomarker to determine the average biomarker copy number per cell over a cell population. These standard cells are then analysed by the single cell analysis technology alone or concomitantly with other samples of interest to determine the single cell copy numbers of all analysed single cells. Typically sets of standard cells that express the biomarker of interest to generate calibration curves for the single cell copy number determination are used. As a result, the current lack of absolute quantification in affinity binder based single cell analysis technologies is overcome, and we thus realize an important measurement parameter which is highly relevant in many clinical, pharmaceutical and research applications.

As a result of this approach, the inventors disclose a list of validated assays for biomarker quantification on the single cell level.

### DETAILED DESCRIPTION OF THE FIGURES

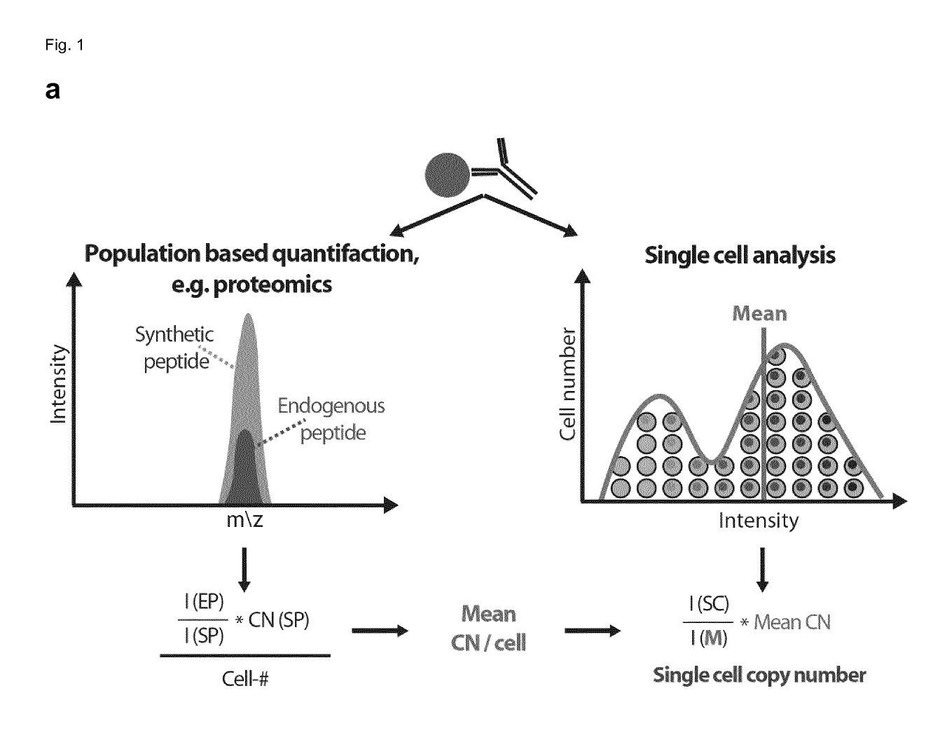

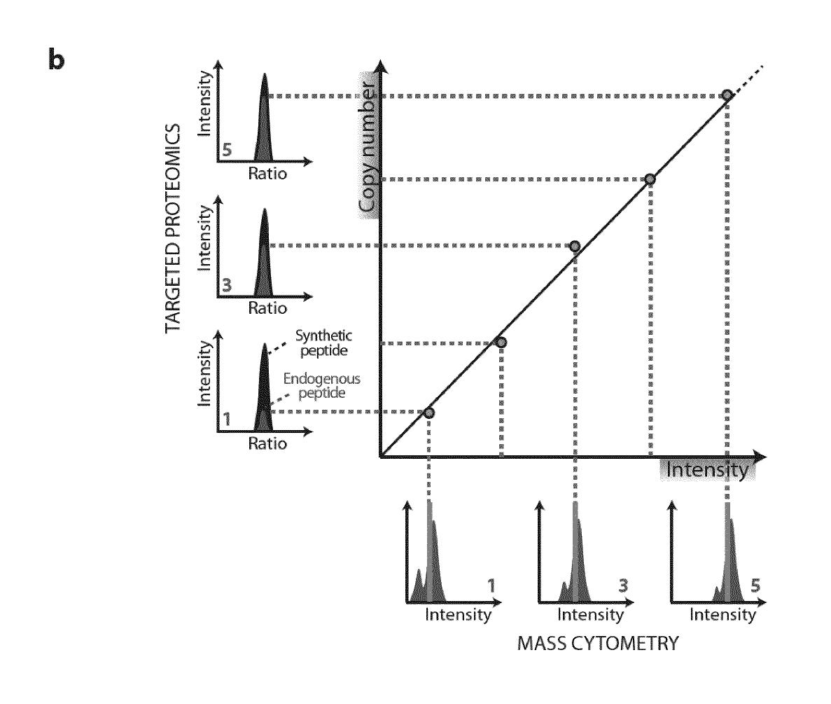

The general concept to determine the absolute number of biomarker molecules on the single cell level is described in FIG. 1. First a mean copy number (CN) per cell is determined with a population based quantification method using a labelled synthetic peptide (SP). In a second step in a single cell analysis method a relative quantity value for the biomarker of interest is determined and calibrated with the mean copy number per cell value to yield a single cell copy number.

To determine the absolute numbers for a biomarker of interest (BOI) first the average copy number per cell is determined using cell population measurement techniques, such as mass spectrometry. These average copy numbers are determined for several cell samples that differ in the abundance of the molecule of interest. Then these cells are analysed using the single cell analysis technique and the mean number of biomarker molecules per cell. Relating the average single copy number with the mean single cell copy number allows to generate a calibration line/curve. Based on this calibration curve/line the single cell number of biomarker molecules can be computed.

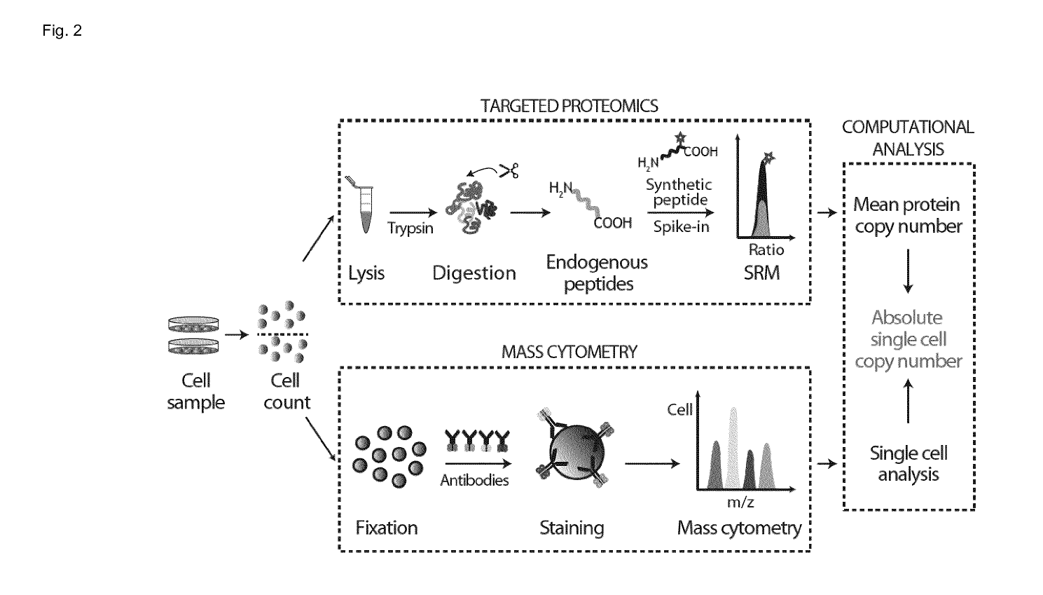

FIG. 2 describes the approach to determine the average single cell copy number for a BOI. Biomarkers are determined for the clinical, biomedical or research question of interest and the corresponding affinity binders are selected. Then, for each BOI peptides are selected that univocally identify and describe the protein or the protein modification. For these peptides then mass spectrometry assays are developed to find and measure those peptides in complex peptide mixtures. In an exemplary set-up, those mass spectrometry assays are for a single reaction monitoring measurement, in which the mass-over-charge ratio (m/z) for the peptide and the m/z ratios of a set of peptide fragments is defined to identify and measure the peptide by LC-MS/SRM (SRM: selected reaction monitoring). To then quantify those peptides in a cell lysate, first proteins are isolated, digested using a protease and a known amount of a synthetic peptide is spiked into the peptide mixture. Then both the intensity of the endogenous and spike-in standard are measured by LC-MS/SRM and the ratio between those two is used to determine the number of the endogenous biomarker peptide molecules. The analysed cells can either be part of the sample that then is analysed by the single cell analysis technology, or can be other cells (standard cells) that then are co-measured with a sample of interest to calibrate the signal of these. In the next step then the cells are prepared for single cell analysis (optionally the standard cells are spiked in), stained with the affinity binders of interest, and are analysed by the single cell analysis technology, e.g. mass cytometry. Based on the signal of the standard cells (that can also be cells which are part of the sample of interest) the mean signal intensity (MSI) is computed. This MSI corresponds to the copy number per cell determined by the cell population assay (SRM analysis). Given that the signal response line or curve is known, the copy number can be computed for each single cell.

In other words, for each BOI peptides that uniquely represent the biomarker (biomarker representing peptide, BRP) in a cell or cell mixture are determined and their abundance is quantified in mass spectrometry. These BRPs can also include and represent protein modifications. For these BRPs, synthetic peptides are synthesized which are chemically identical, but differ in the mass. This is achieved by incorporation of heavy, stable isotopes during peptide synthesis. To determine the abundance of the biomarker via the BRP, a cell sample is split, and part is lysed and peptides are generated via enzymatic digestion. Then the synthetic, isotopically labelled version of the BRP, of which the exact number of molecules is known, is spiked into the peptide sample and concomitantly measured with the BRP. The ratio of labelled and endogenous peptide together with the known starting cell number is used to compute the average number of BOI molecules per cell in the analyzed sample.

The cell sample left after splitting then is used as a standard in the single cell measurements for the BOI. The BOI is labelled using a reporter carrying affinity binder and the signal is analysed by a single cell analysis technology. After such a measurement, the mean signal of the biomarker over all analysed single cells can be computed. This computed mean is equal to the measured mean in the population-based measurement (i.e. mass spectrometry) and thus the average number of biomarker molecules per cell can be assigned to it. This then allows to compute a calibration curve for single cell copy number determination as described above.

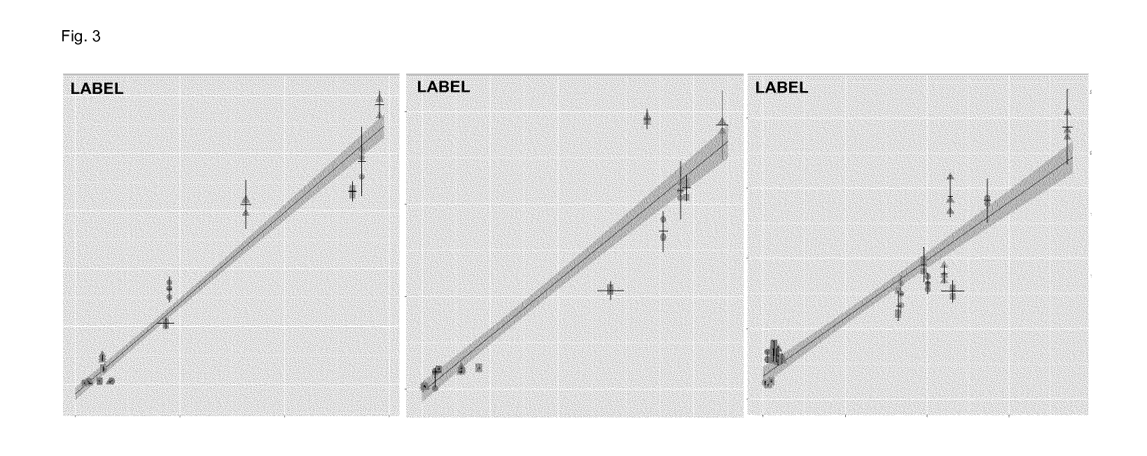

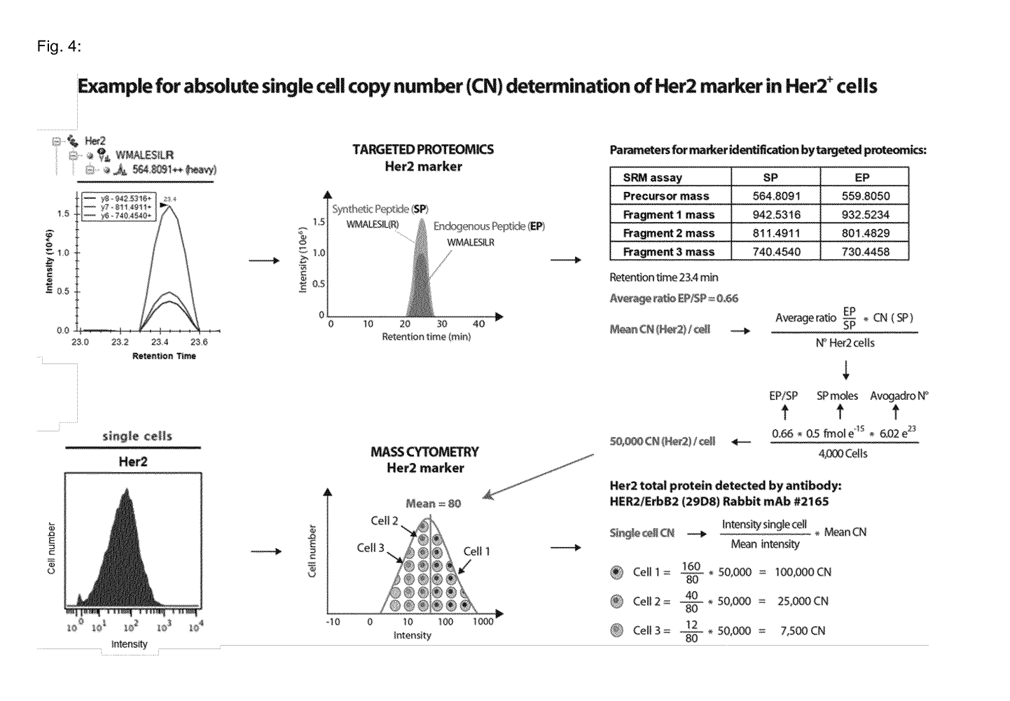

FIG. 3 shows example calibration curves for CD44, vimentin and c-Met, respectively. The calibration curve values are y=−172256+31482x for CD44, y=756668+24156x for vimentin and y=32692+412x for c-Met. The x-axis shows mass cytometry ion counts, the y-axis shows average single cell copy numbers.

Technical Specifications

Samples

The disclosed invention can be applied to a variety of samples to determine the absolute number of biomarker molecules per single cell. These samples include cells in suspension, cells on surfaces and cells in a three dimensional context such as in a tissues. The cells can come from cells grown in culture, cells from any tissue and can be from any organism.

To determine the absolute number of biomarker molecules cells can either be in suspension form, in a single cell layer as analysed in immunocytochemical applications, and in the form of sections (in the case of tissues) in immunocytochemical applications. Also, cells in tissues can be dissociated to generate single cell suspensions in order to determine the absolute copy numbers.

Single Cell Analysis Technologies

A variety of single cell analysis technologies exist, that rely on the detection of biomarkers using affinity binders—all can be applied to determine the absolute number of biomarker molecules on the single cell level with the instant invention. These methods include flow cytometry, mass cytometry, immunohistochemistry, immunocytochemistry, variants of microscopy, microfluidic devices and combinations thereof. In a preferred embodiment, mass cytometry is used to determine single cell absolute quantities.

Affinity Binders

The affinity binders that can be used with the approach include antibodies and parts thereof (e.g. single chain antibody fragments), RNA/DNA binders such as aptamers and variants thereof and DARPINs (Designed Ankyrin Repeat Proteins). The affinity binders can be coupled to a wide range of reporters, including but not limited to fluorophores, pure isotopes, elements with a natural isotopic distribution, an isotope mixture with a defined ratios of the isotopes, DNA, RNA and molecules with a defined mass over charge ratio (m/z) in mass spectrometric applications.

Staining/Labeling of Cells with Affinity Binders

The staining of single cells follows standard staining protocols known in the art for binding reagents, such as antibodies, to cells.

Biomarker Representing Peptide (BRP)

The BRP to calibrate the single cell analysis epitope signal has a defined and unique m/z allowing its univocal identification. The BRP fragment m/z values and/or their relative intensity are used with the peptides m/z value for its identification. To determine the ratio between the BRP and the BOI peptide, the peptide fragment ion intensities and/or the peptide ion intensity is used. A wide variety of methods can be used to analyse and quantify the peptides using MS. First, the peptides have to be ionized. To ionize the peptide, any of the following methods can be used: electron and chemical ionization, spray ionization (e.g. electrospray ionization), desorption ionization (e.g. matrix-assisted laser desorption ionization), gas discharge ionization, ambient ionization and any other used methods to ionize analytes for MS.

The fragments of the peptide can be generated by collision induced dissociation, infrared multiphoton dissociation, blackbody infrared radiative dissociation, electron-capture dissociation, (negative) electron-transfer dissociation, electron-detachment dissociation, surface induced dissociation and combinations and variants thereof.

Appropriate MS instruments to determine the m/z and intensity of the peptides are time of flight (TOF), quadrupole, ion trap, fourier transform ion cyclotron resonance, orbitrap, sector field, any other mass analyser and combinations thereof.

One MS instrument set-up which is particularly suitable to measure and quantify the peptides for absolute quantification due to its precision and sensitivity is a triple quadrupole MS instrument. It achieves low attomole sensitivity, allows detection of 1, 2, 3 or at least 5, or at least 10, or at least 50, or at least 100, or at least 200, or at least 300, or at least 500, or at least 1000 peptides in a single analysis via collision induced dissociation (CID) coupled to liquid chromatography.

The synthetic peptides to perform the absolute quantification are synthesized with defined isotopes of any existing element with 1 or n Dalton mass differences that allow to uniquely identify and quantify them in a MS measurement. Ultimately, the minimal needed mass difference will be defined by the achievable resolution of the MS instrument. Suitable elements with their isotopes include without being limited to hydrogen, carbon, nitrogen, oxygen, sulphur, chlorine, fluorine and bromide.

Standard Cells

To generate the protein lysate from the standard cells and subsequently peptides from protein, the following methods can be used: chemical, mechanical, enzymatic, sonic and electromagnetic methods.

In a certain embodiment, cells are lysed using mechanical force and are digested using the trypsin protease. Other proteases to digest the protein into peptides include LysC, Asp-N, Glu-C, Lys-C, Arg-C, pepsine, chemotrypsin, any other protease and combinations thereof.

The peptides are either unmodified or modified. In a certain embodiment the peptides are unmodified or phosphorylated on serine, threonine, tyrosine, histidine, aspartate and glutamate residues. Other modifications on any other amino acid residue of the peptides can include (Z)-2,3-didehydrotyrosine,1-thioglycine, 2,3-didehydroalanine (Ser), 2,3-didehydrobutyrine, 2′,4′,5′-topaquinone, 2-oxobutanoic acid, 3-oxoalanine (Cys, Ser), 3-phenyllactic acid, acetylation, acid aspartate ester, ADP-ribosylation, allysine, amidation, beta-methylthiolation, biotin, bromination, cholesterol, cis-14-hydroxy-10,13-dioxo-7-heptadecenoic, citrullination, C-Mannosylation, cysteine persulfide, cysteine sulfenic acid (—SOH, —SO2H), deamidation, deamidation followed by a methylation, dihydroxylation, dimethylation, dimethylation of proline, diphthamide, FAD, FMN conjugation (Cys, His, Ser/Thr), formylation, gamma-carboxyglutamic acid, geranyl-geranylation, glucosylation (Glycation), glutathionylation, hydroxylation, hypusine, lipoyl, methionine sulfone, methylation, myristoylation, N6,N6,N6-trimethyl-5-hydroxylysine, N6-1-carboxyethyl lysine, N6-poly(methylaminopropyl)lysine, n-Decanoate, n-Octanoate, O-GlcNAc, Omega-hydroxyceramide glutamate ester, palmitoylation, phosphatidylethanolamine amidated glycine, phosphopantetheine, phosphorylation, pyridoxal phosphate, pyrrolidone carboxylic acid, pyrrolidone carboxylic acid (Glu), pyrrolysine, pyruvic acid (Cys), pyruvic acid (Ser), S-12-hydroxyfarnesyl cysteine, S-archaeol, S-diacylglycerol cysteine, S-farnesyl cysteine, S-Nitrosylation, S-palmitoleyl cysteine, sulfation, thyroxine, triiodothyronine and trimethylation.

Besides peptides, other molecules that can be quantified by mass spectrometry and mass cytometry or other single cell analysis techniques can be absolutely quantified using the presented approach here. These include small molecule compound, nucleotide, DNA, RNA, metabolite, mono-saccharide, poly-saccharide, metalo-organic compound and any combination of the above mentioned.

1. A method for determining the number of molecules of a biomarker, said method comprising the steps of:
a) obtaining a first portion of a cell population, wherein said cell population comprises a plurality of single cells characterized by the presence of said biomarker,
b) obtaining a cell number, c(1), of said first portion,
c)
i. subjecting the cells of said first portion to conditions whereby said biomarker is fragmented, yielding a biomarker fragment, in a fragmentation step;
ii. adding to said biomarker fragments a known number n(k) of labelled biomarker fragments differing from said biomarker fragment only in a detectable label,
iii. measuring a first and a second parameter of said first portion, wherein said first parameter corresponds to the amount of said biomarker fragment and said second parameter corresponds to the amount of said labelled biomarker fragment yielding a biomarker fragment value, v(u), and a labelled biomarker fragment value v(k), respectively, and
iv. in a first computational step, relating v(u) and v(k) with n(k) and c(1), thereby determining an average number of biomarker molecules per cell, m(u), of said first portion,
d) repeating the steps under b) and c) for sets of cells that express the biomarker at different levels, generating a set of values m(u)1 . . . n then
e)
v. subjecting the cells of a second portion of said cell population to conditions whereby a labelled affinity binder specifically binds to said biomarker,
vi. measuring a value of said bound labelled affinity binder for a plurality of said cells of said second portion, wherein said value corresponds to the amount of said biomarker, yielding a single cell measurement value s(u) for a plurality of cells of said second portion, and determining from said single cell measurement value a mean measurement value m(s) for said plurality of cells, and
vii. performing step v and vi) for said sets of cells that express the biomarker at different levels,
viii. relating, in a second computational step, the mean measurement values m(s)1 . . . n and the average number of biomarker molecules per cell, m(u)1 . . . n and computing a calibration curve,
ix. relating s(u) with the calibration curve yielding a number of biomarker molecules, n(u), for each single cell of said plurality of single cells.

2. A method for determining the number of molecules of a biomarker, said method comprising the steps of:
a) obtaining a first portion of a cell population, wherein said cell population comprises a plurality of single cells characterized by the presence of said biomarker,
b) obtaining a cell number, c(1), of said first portion,
c)
i. subjecting the cells of said first portion to conditions whereby said biomarker is fragmented, yielding a biomarker fragment, in a fragmentation step;
ii. adding to said biomarker fragments a known number n(k) of labelled biomarker fragments differing from said biomarker fragment only in a detectable label,
iii. measuring a first and a second parameter of said first portion, wherein said first parameter corresponds to the amount of said biomarker fragment and said second parameter corresponds to the amount of said labelled biomarker fragment yielding a biomarker fragment value, v(u), and a labelled biomarker fragment value v(k), respectively, and
iv. in a first computation step, relating v(u) and v(k) with n(k) and c(1), thereby determining an average number of biomarker molecules per cell, m(u), of said first portion,
d)
v. subjecting the cells of a second portion said cell population to conditions whereby a labelled affinity binder specifically binds to said biomarker,
vi. measuring a value of said bound labelled affinity binder for a plurality of said cells of said second portion, wherein said value corresponds to the amount of said biomarker, yielding a single cell measurement value s(u) for a plurality of cells of said second portion, and determining from said single cell measurement value a mean measurement value m(s) for said plurality of cells, and
vii. in a second computation step, relating, s(u), m(s) and m(u), yielding a number of biomarker molecules, n(u), in each of said plurality of single cells.

3. The method according to claim 1, wherein the first computation step is calculated using formula I:
$$\frac{\frac{v ( u )}{v ( k )} * n ( k )}{c ( 1 )} = m ( u ) .$$

4. The method according to claim Error! Reference source not found, wherein the second computation step is calculated using the formula:
$$\frac{s ( u )}{m ( s )} * m ( u ) + d ( s ) = n ( u )$$
wherein the term d(s) is the lowest detectable copy number of a molecule.

5. The method according to claim 4, wherein d(s) is determined by analysing separate cell samples known to express different levels of the biomarker, particularly wherein a calibration curve or -line is determined yielding d(s), or wherein d(s) is determined based on the measurement of a single cell sample, the detection limit of the instrument and the average signal expected per affinity binder.

6. The method according to claim 1, wherein for the second computation step the ratio between s(u) and m(u) is defined by a response function defined by the instrument response (and/or antibody binding behaviour) over the measured dynamic range (non-linear calibration curve).

7. A method for quantifying a biomarker on a single cell level, comprising the steps of:
a) providing a first cell population comprising said single cells, wherein said single cells comprise said biomarker,
b) taking a first and a second portion of said cell population and obtaining the cell number of the first portion,
c)
i. adding a known number of molecules of a labelled biomarker or fragments thereof to said first portion,
ii. obtaining a relative quantity value for said biomarker or fragment thereof and said labelled biomarker or fragment thereof in said first portion, and
iii. relating the relative quantity of said biomarker or fragment thereof and said labelled biomarker or fragment thereof with said known number of molecules of the labelled biomarker or fragment thereof and said number of cells of said first portion yielding an average number of biomarker molecules per cell in said first portion;
d)
iv. subjecting the cells of said second portion to conditions whereby a labelled affinity binder specifically binds to said biomarker,
v. measuring a value of said bound labelled affinity binder for a plurality of cells of said second portion, wherein said value corresponds to the amount of said biomarker, yielding a single measurement value for a plurality of cells of said second portion, and determining from said single measurement value a mean measurement value for said plurality of cells, and
e) repeating the steps under b), c) and d) for sets of cells that express the biomarker at different levels,
f) creating a calibration curve relating the average number of biomarker molecules to the mean measurement value for a number of different cell populations,
g) relating said single measurement value, said mean measurement value and said average number of biomarker molecules per cell, yielding a number of biomarker molecules in said single cell.

8. (canceled)

9. The method according to claim 1, wherein the biomarker is a peptide derived from a protein, a peptide, an organic small molecule compound, a DNA molecule, an RNA molecule, a oligoribonucleotide, a mono-sachharide, a poly-saccharide or a metalo-organic compound.

10. The method according to claim 1, wherein the biomarker is a posttranslationally modified peptide.

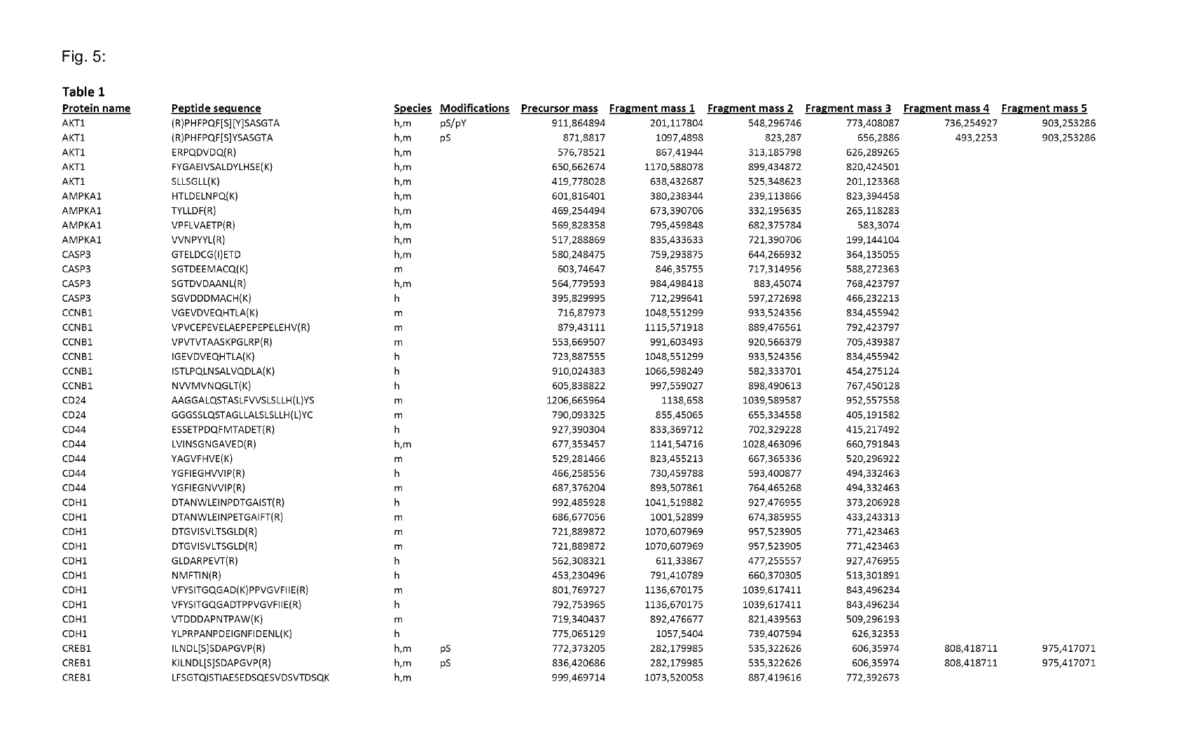

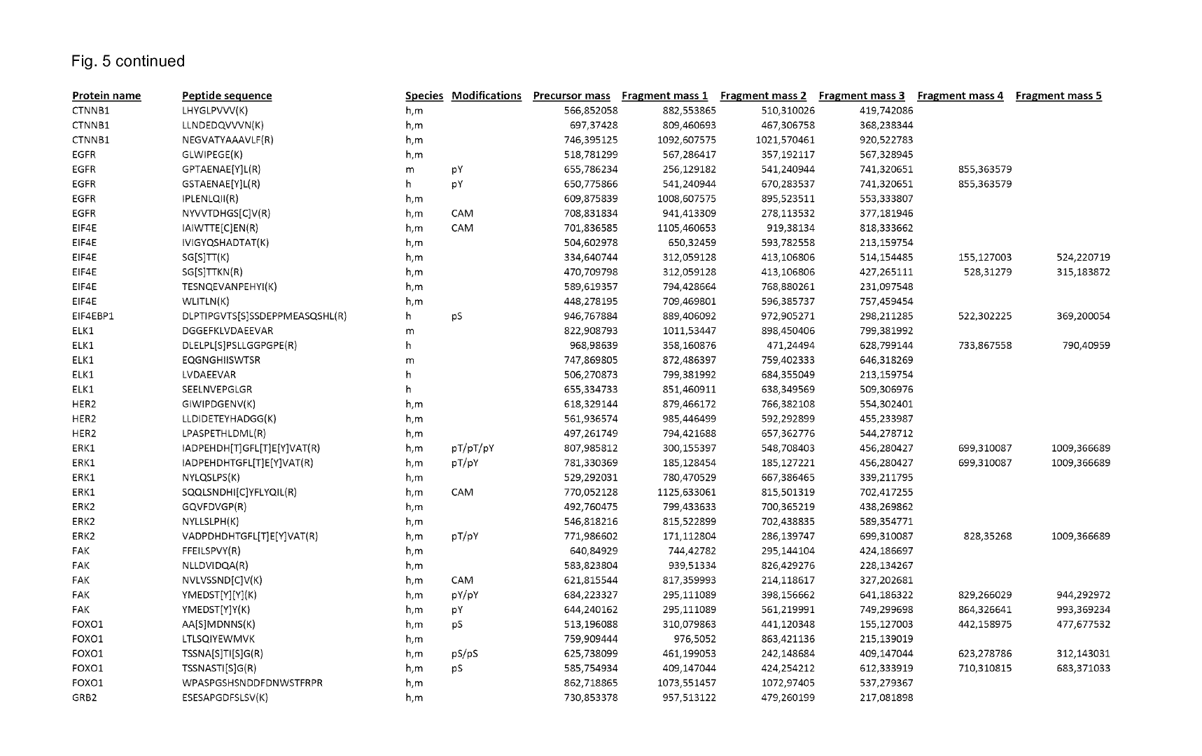

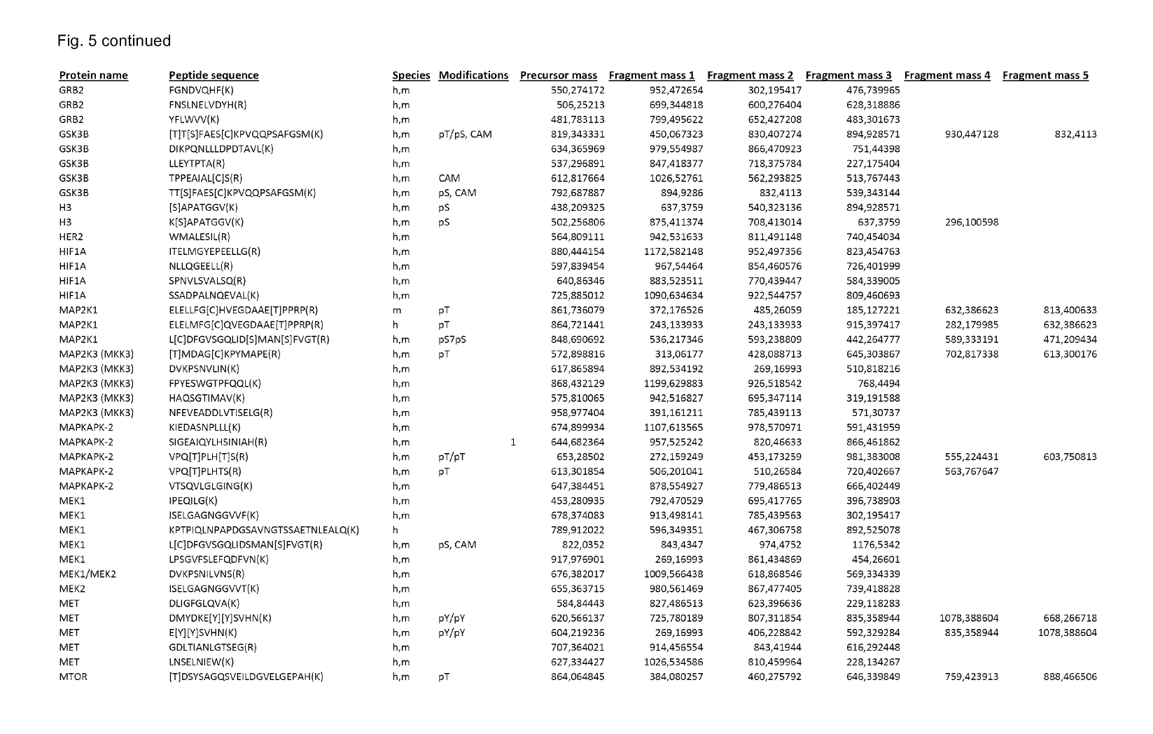

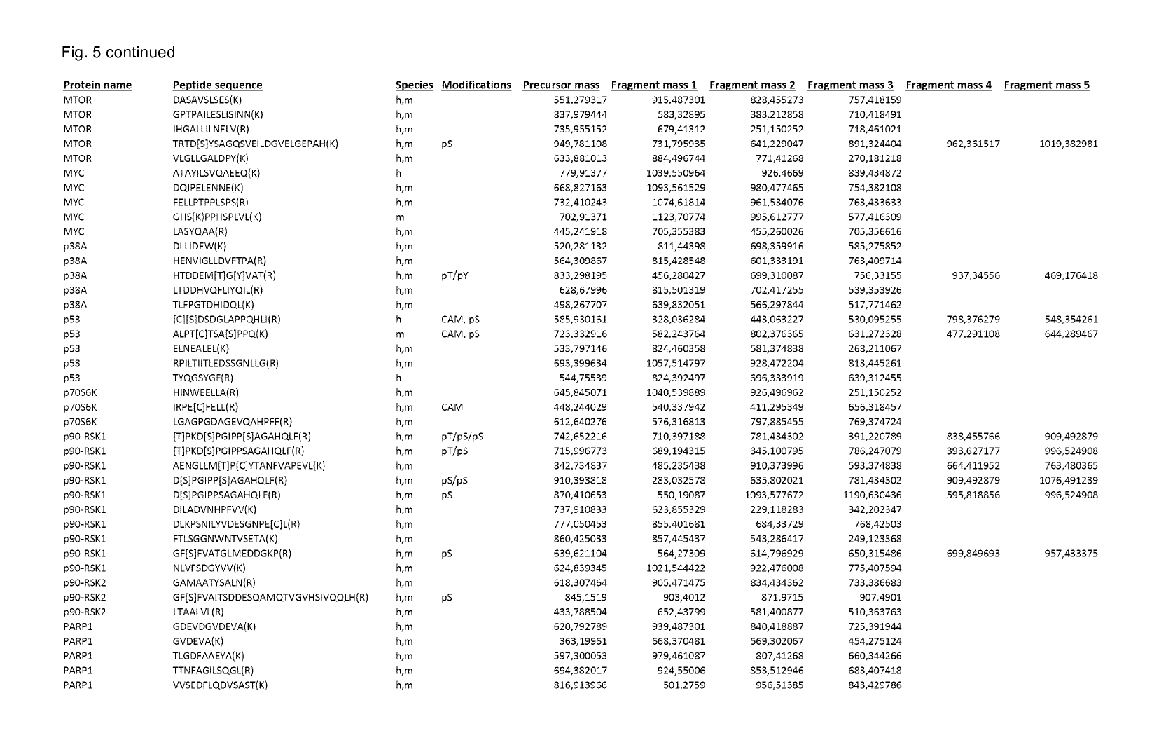

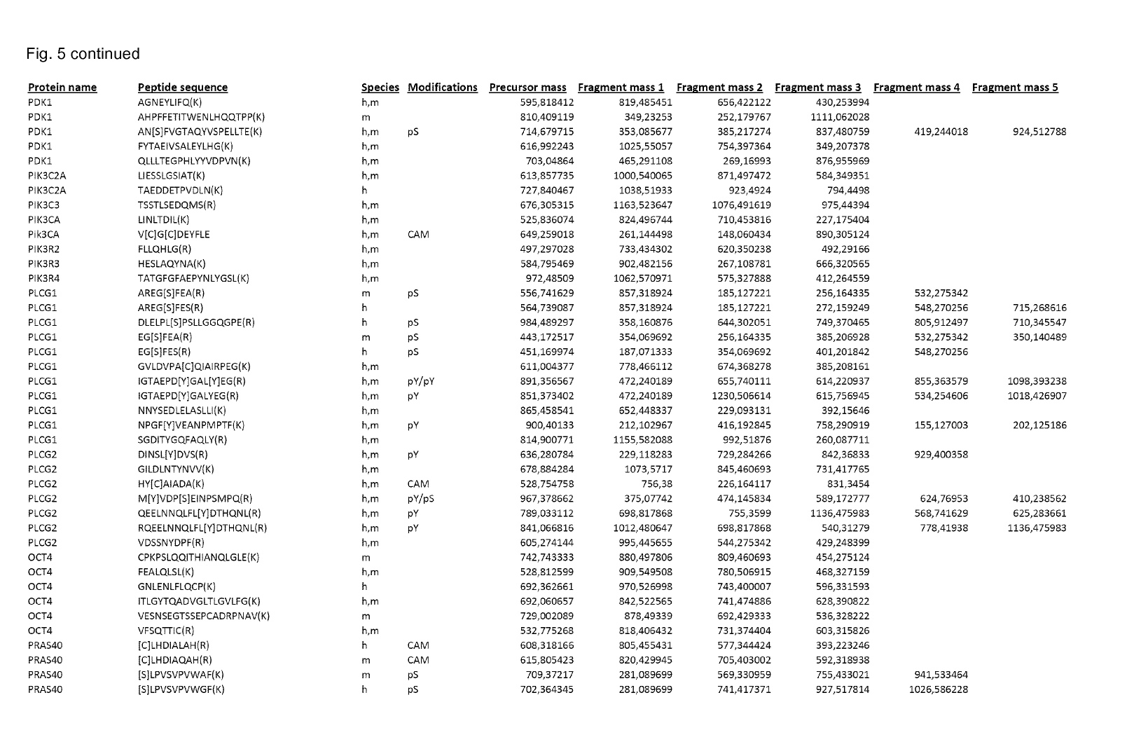

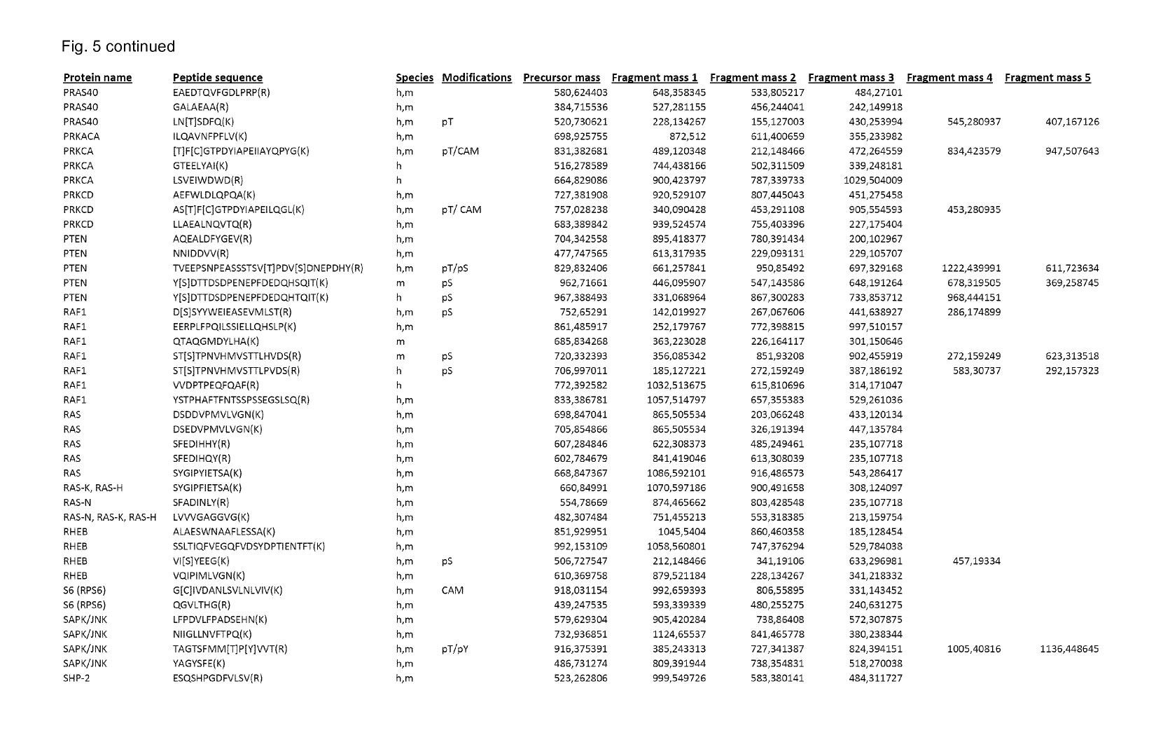

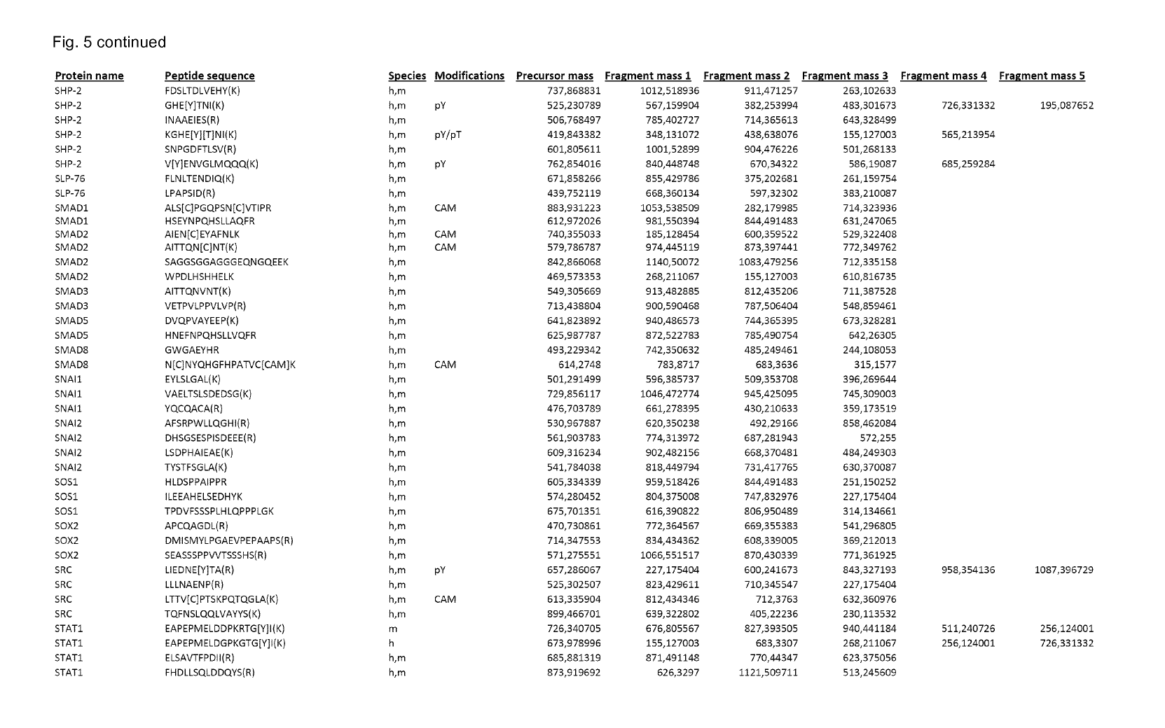

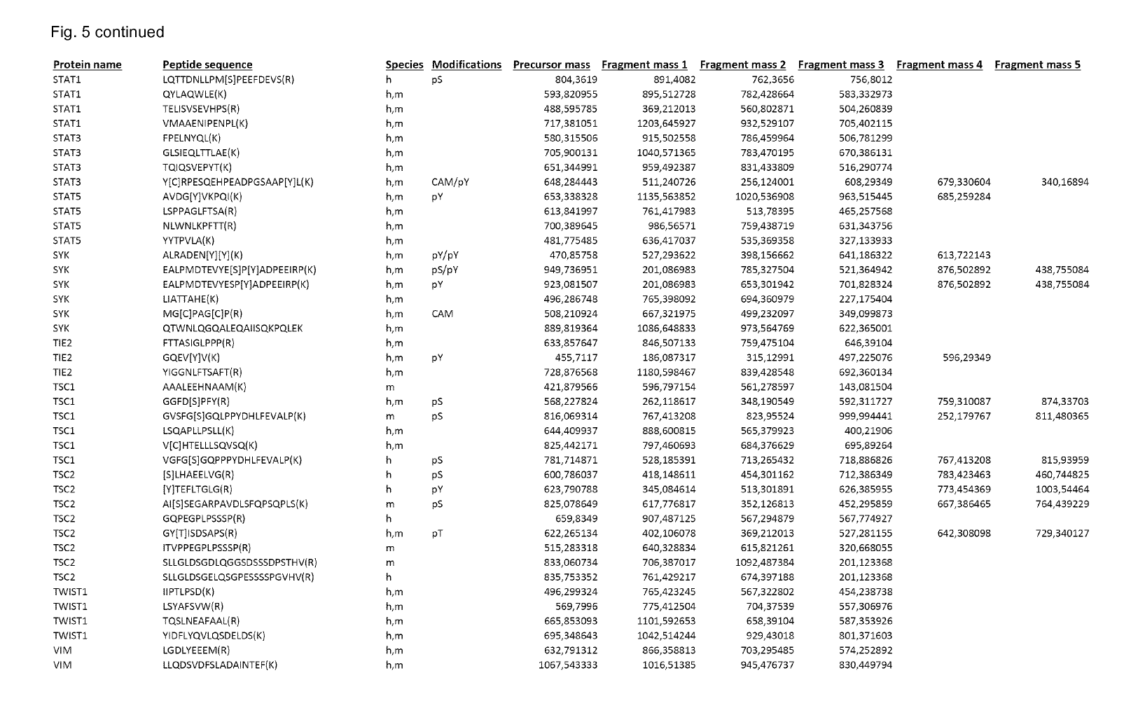

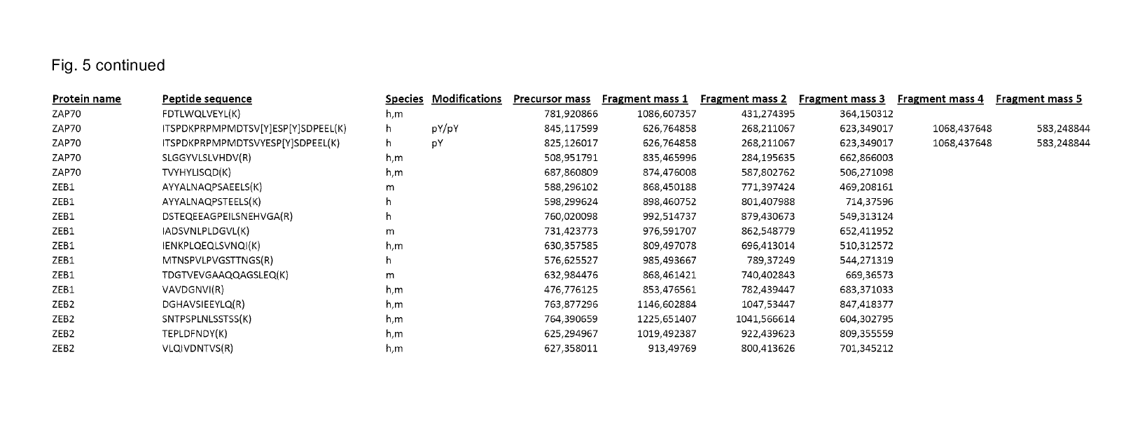

11. The method according to claim 1, wherein the amino acid sequence of said biomarker fragment is selected from the second column in the table shown in FIG. 5.

12. The method according to claim 1, wherein the method of fragmentation in the fragmentation step is enzymatic digestion.

13. The method according to claim 1, wherein the biomarker is fragmented to yield a plurality of biomarker fragments (fragment 1, fragment 2, fragment n) in the fragmentation step, and a known number n(k1), n(k2), n(k3) of labelled biomarker fragments is added for each biomarker fragment in step ii.

14. The method according to claim 1, wherein the biomarker fragments resulting from the fragmentation step are subjected to mass spectrometry, based methods particularly collision induced dissociation, infrared multiphoton dissociation, blackbody infrared radiative dissociation, electron-capture dissociation, negative electron-transfer dissociation, electron-detachment dissociation or surface induced dissociation.

15. The method according to claim 1, wherein the labelled biomarker fragment is labelled with a stable isotope.

16. The method according to claim 1, wherein said first and second parameter are MS1 mass spectrometry signal intensity values at a given m/z value or given m/z values, or MS2 peptide fragment intensity values at a single or multiple m/z value, or MS3 peptide fragment intensity values at a single or multiple m/z value.

17. The method according to claim 1, wherein said first and said second parameter can be the intensity and m/z values, and/or the retention time constraint of a fragment in a chromatographic system or a combination thereof.

18. The method according to claim 1, wherein said value is selected from fluorescence intensity value, mass spectrometry signal intensity value, spectrophotometric values, western blot intensity values, RNA sequencing values and quantitative PCR values.

19. The method according to claim 1, wherein the labelled affinity binder is selected from an antibody, RNA/DNA binder particularly aptamers and a DARPINs (designed ankyrin repeat proteins).

20. The method according to claim 1, wherein the labelled affinity binder is labelled with a fluorophore, a stable marker isotope, a DNA or RNA marker, a protein marker, an enzyme marker or any other marker, which particularly may be detectable by a second marker.

21. The method according to claim 1, wherein the number of biomarker molecules is determined for a plurality of different biomarkers.

22. (canceled)

23. (canceled)
